(19) United States
(12) Patent Application Publication
(10) Pub. No.: US 2008/0262066 A1
(43) Pub. Date: Oct. 23, 2008

(54) Azole Derivatives as Cannabinoid CB1 Receptor Antagonists

(75) Inventors: Jinhwa LEE, Yongin-si (KR); Jeongmin Kim, Yongin-si (KR); Kwang-Seop Song, Yongin-si (KR); Min Ah Kim, Yongin-si (KR); Jong Yup Kim, Yongin-si (KR); Suk Ho Lee, Yongin-si (KR); Hee Jeong Seo, Yongin-si (KR); Sung-Han Lee, Yongin-si (KR); Myung Eun Jung, Yongin-si (KR); Kwang Woo Ahn, Yongin-si (KR); Eun Jung Son, Yongin-si (KR)

Correspondence Address: SUGHRUE MION, PLLC, 2100 PENNSYLVANIA AVENUE, N.W., SUITE 800, WASHINGTON, DC, 20037, US

(73) Assignee: Green Cross Corporation, Yongin-si (KR)

(21) Appl. No.: 12/105,460

(22) Filed: Apr. 18, 2008

Related U.S. Application Data
(60) Provisional application No. 60/913,041, filed on Apr. 20, 2007.

(51) Int. Cl. A61K 31/415; C07D 231/14; A61K 31/4164; A61P 3/00; C07D 233/54

(52) U.S. Cl. 514/400; 548/374.1; 548/333.5; 514/406

(57) ABSTRACT

A novel azole compound of formula (I) or a pharmaceutically acceptable salt thereof is effective as a cannabinoid CB₁ receptor inverse agonist or antagonist, which is useful for preventing or treating obesity and obesity-related metabolic disorders. The prevention also provides a method for preparing same, a pharmaceutical composition containing same, and a method for preventing or treating obesity and obesity-related metabolic disorders.

### FIELD OF THE INVENTION

The present invention relates to a novel azole compound which is effective as a cannabinoid CB₁ receptor inverse agonist or antagonist.

### BACKGROUND OF THE INVENTION

The World Health Organization (WHO) recently reported that obesity has become a global epidemic, posing a serious threat to public health because of the increased risk of associated health problems (See Report of a WHO Consultation on Obesity: Obesity-Preventing and Managing a Global Epidemic; World Health Organization: Geneva, 1997). Obesity is characterized by excess body fat, especially visceral fat, and constitutes a pro-inflammatory state eventually leading to serious health consequences. There are growing evidences that obesity as a chronic disease cannot be cured by short-term dieting or exercise alone, but additional pharmacological treatments would lead to higher success rates.

CB₁ cannabinoid receptor belongs to G-protein-coupled receptor (GPCR) type and is coupled to inhibitory G proteins (G(i/o)) to inhibit certain adenylyl cyclase isozymes, leading to decreased cAMP production, decreased Ca²⁺ conductance, increased K⁺ conductance, and increased mitogen-activated protein kinase activity (See Di Marzo et al., Nat. Rev. Drug Discovery 2004, 3, 771-784; Rhee, M. H. et al., J. Neurochem. 1998, 71, 1525-1534). The major physiological effect of cannabinoids (in the central nervous system (CNS) and neuronal tissues) is the modulation of neurotransmitter release via activation of presynaptic CB₁ receptors located on distinct types of axon terminals throughout the brain (See Howlett, A. C. et al., Neuropharmacology 2004, 47 (Suppl. 1), 345-358).

The CB₁ receptor is mainly expressed in several brain areas including the limbic system (amygdala, hippocampus), hypothalamus, cerebral cortex, cerebellum, and basal ganglia. In the cerebellum and basal ganglia cannabinoids modulate the locomotor activity. In the limbic system, cannabinoids influence learning, memory, emotion, and motivation, and through activation of CB₁ receptors in the limbic system-hypothalamus axis, cannabinoids have an important role in the control of appetite. Moreover, lower levels of CB₁ receptors can also be found in peripheral tissues including urinary bladder, testis, prostate, GI tract, heart, lung, adrenal gland, parotid gland, bone marrow, uterus, ovary, and adipose tissue (See Cota, D. et al., J. Clin. Invest. 2003, 112, 423-431; Ravinet Trillou, C. et al., Int. J. Obes. Relat. Metab. Disord. 2004, 28, 640-648; Galiegue, S. et al., Eur. J. Biochem. 1995, 232, 54-61; Howlett, A. C. et al., Pharmacol Rev. 2002, 54, 161-202).

Many preclinical in vitro and in vivo experiments have been shown that CB₁ receptor antagonists can influence energy homeostasis by central and peripheral mechanisms and may represent promising targets to treat diseases that are characterized by impaired energy balance. Already the first published studies with rimonabant (SR141716) in both rodents (See Arnone, M. et al., Psychopharmacology (Berlin) 1997, 132, 10-106) and primates (See Simiand, J.; Keane, M.; Keane, P. E.; Soubrie, P. Behav. Pharmacol. 1998, 9, 179-181) showed clear differentiation, i.e., marked effects on sweet food intake versus marginal effects on regular chow intake or water drinking. Many other preclinical “proof of concept” studies have been performed in the meantime with several CB agonists and antagonists to further uncover the amount and mode of contribution of cannabinergic system modulators to energy homeostasis. Almost all of those studies have been recently reviewed (See Smith, R. A. et al., IDrugs 2005, 8, 53-66).

Considering the important impact of obesity on public health and the lack of any efficient and viable drug to cure it, it is no surprise that CB₁ antagonists are currently the subject of intense studies, which were published in several reviews (See Adam, J. et al., Expert Opin. Ther. Patents, 2002, 12(10), 1475-1489; Hertzog, D. L. Expert Opin. Ther. Patents, 2004, 14(10), 1435-1452; Lange, J. H. M. et al., Drug Discov. Today, 2005, 10, 693-702; Bishop, M. J. J. Med. Chem., 2006, 49(14), 4008-4016).

### SUMMARY OF THE INVENTION

It is a primary object of the present invention to provide a novel azole compound of formula (I) or a pharmaceutically acceptable salt thereof, which is effective as a cannabinoid CB₁ receptor inverse agonist or antagonist, useful for preventing or treating obesity and obesity-related metabolic disorders.

It is another object of the present invention to provide a method for preparing the inventive compound.

It is another object of the present invention to provide a pharmaceutical composition for preventing or treating obesity and obesity-related metabolic disorders, comprising the inventive compound as an active ingredient.

### DETAILED DESCRIPTION OF THE INVENTION

In accordance with one aspect of the present invention, there is provided a compound of formula (I) or a pharmaceutically acceptable salt thereof and a method for preparing same:

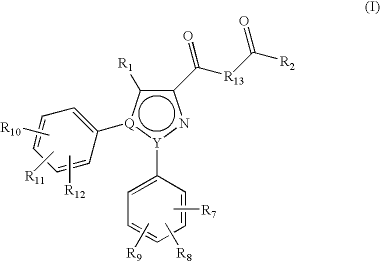

wherein:

Q is carbon and Y is nitrogen, or Q is nitrogen and Y is carbon;

R₁ is hydrogen, halogen, C₁₋₇ alkyl, substituted C₁₋₇ alkyl, C₂₋₇ alkenyl, substituted C₂₋₇ alkenyl, C₂₋₇ alkynyl, substituted C₂₋₇ alkynyl, or (CH₂)ₙ—C₃₋₅ carbocycle, n being 0 or 1;

R₂ is hydrogen, OR₃, NR₄R₅, C₁₋₇ alkyl, substituted C₁₋₇ alkyl, C₂₋₇ alkenyl, substituted C₂₋₇ alkenyl, C₂₋₇ alkynyl, substituted C₂₋₇ alkynyl, C₃₋₇ cycloalkyl, substituted C₃₋₇ cycloalkyl, aryl, substituted aryl, heteroaryl, substituted heteroaryl, heterocycloalkyl, substituted heterocycloalkyl; C₁₋₈ alkyl optionally substituted by alkoxy or halogen, C₂₋₆ alkenyl optionally substituted by alkoxy or halogen, (CH₂)ₘ—C₃₋₆ carbocycle optionally substituted by alkoxy or halogen, or (CH₂)ₘ—R₆, m being 1 or 2;

R₃ is C₁₋₇ alkyl, substituted C₁₋₇ alkyl, C₂₋₇ alkenyl, substituted C₂₋₇ alkenyl, C₃₋₇ cycloalkyl, substituted C₃₋₇ cycloalkyl, aryl, substituted aryl, heteroaryl, substituted heteroaryl, heterocycloalkyl, substituted heterocycloalkyl;

R₄ and R₅ are each independently hydrogen, C₁₋₆ alkyl, substituted C₁₋₆ alkyl, C₂₋₆ alkenyl, substituted C₂₋₆ alkenyl, C₃₋₇ cycloalkyl, substituted C₃₋₇ cycloalkyl, aryl, substituted aryl, heteroaryl, substituted heteroaryl, heterocycloalkyl, substituted heterocycloalkyl; or

R₄ and R₅, together with the nitrogen atom to which they are bonded, form a 4- to 10-membered saturated or unsaturated heterocyclic ring which is optionally substituted with one or more C₁₋₃ alkyl, benzyl, phenyl, C₁₋₃ alkoxy or halogen;

R₆ is phenyl, furanyl, benzofuranyl, thienyl, benzothienyl, pyridinyl, pyrimidinyl, pyrazinyl, pyridizinyl, tetrahydrofuranyl, tetrahydropyranyl, dioxanyl, 1,4-benzodioxanyl or benzo[1,3]dioxolyl, each being optionally substituted by one or more groups consisting of halogen, C₁₋₃ alkyl and C₁₋₂ alkoxy, each having optional one to three fluorine substitutes;

R₇, R₈, R₉, R₁₀, R₁₁ and R₁₂ are each independently hydrogen, halogen, cyano, C₁₋₃ alkyl, C₁₋₃ alkoxy or trifluoromethyl; and

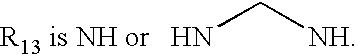

As used herein, the term “alkyl” refers to a straight or branched chain saturated hydrocarbon radical. Examples of “alkyl” as used herein include, but are not limited to, methyl, ethyl, n-propyl, isopropyl, n-butyl, isobutyl, t-butyl, n-pentyl, isopentyl and hexyl.

As used herein, the term “substituted alkyl” refers to a straight or branched chain saturated hydrocarbon radical, which is optionally substituted by one or more substituents selected from the group consisting of C₁₋₃ alkyl optionally having one to three fluorine substituents, C₂₋₃ alkenyl, C₂₋₃ alkynyl, C₁₋₂ alkoxy optionally having one to three fluorine substituents, sulfanyl, sulfinyl, sulfonyl, oxo, hydroxy, mercapto, amino, guanidino, carboxy, aminocarbonyl, aryl, aryloxy, heteroaryl, heteroaryloxy, heterocyclic, aminosulfonyl, sulfonylamino, carboxyamide, ureido, nitro, cyano and halogen.

As used herein, the term “alkenyl” refers to a straight or branched chain hydrocarbon radical having at least one carbon-carbon double bond. Examples of “alkenyl” as used herein include, but are not limited to, ethenyl and propenyl.

As used herein, the term “substituted alkenyl” refers to a straight or branched chain hydrocarbon radical having at least one carbon-carbon double bond, which has optional substituents selected from the group consisting of C₁₋₃ alkyl optionally having one to three fluorine substituents, amino, aryl, cyano and halogen.

As used herein, the term “alkynyl” refers to a straight or branched chain hydrocarbon radical having at least one carbon-carbon triple bond. Examples of “alkynyl” as used herein include, but are not limited to, acetylenyl and 1-propynyl.

As used herein, the term “substituted alkynyl” refers to a straight or branched chain hydrocarbon radical having at least one carbon-carbon triple bond, optionally having one or more substituents selected from the group consisting of C₁₋₃ alkyl optionally having one to three fluorine substituents, amino, aryl and halogen.

As used herein, the term “halogen” refers to fluorine (F), chlorine (Cl), bromine (Br), or iodine (I).

As used herein, the term “carbocycle” refers to a non-aromatic cyclic hydrocarbon radical composed of three to seven carbon atoms. Five- to seven-membered rings may contain a double bond in the ring structure. Exemplary “carbocycle” groups include, but are not limited to, cyclopropyl, cyclobutyl, cyclopentyl, cyclopentenyl, cyclohexyl and cycloheptyl.

As used herein, the term “substituted carbocycle” refers to a non-aromatic cyclic hydrocarbon radical composed by three to seven carbon atoms, which is optionally substituted with one or more substituents selected from the group consisting of C₁₋₃ alkyl optionally having one to three fluorine substituents, C₂₋₃ alkenyl, C₂₋₃ alkynyl, C₁₋₂ alkoxy optionally having one to three fluorine substituents, sulfanyl, sulfinyl, sulfonyl, oxo, hydroxy, mercapto, amino, guanidino, carboxy, aminocarbonyl, aryl, aryloxy, heteroaryl, heterocyclic, aminosulfonyl, sulfonylamino, carboxyamide, nitro, ureido, cyano and halogen.

As used herein, the term “aryl” refers to an optionally substituted benzene ring or refers to a ring system which may result by fusing one or more optional substituents. Exemplary optional substituents include substituted C₁₋₃ alkyl, substituted C₂₋₃ alkenyl, substituted C₂₋₃ alkynyl, heteroaryl, heterocyclic, aryl, alkoxy optionally having one to three fluorine substituents, aryloxy, aralkoxy, acyl, aroyl, heteroaroyl, acyloxy, aroyloxy, heteroaroyloxy, sulfanyl, sulfinyl, sulfonyl, aminosulfonyl, sulfonylamino, carboxyamide, aminocarbonyl, carboxy, oxo, hydroxy, mercapto, amino, nitro, cyano, halogen, or ureido.

Such a ring or ring system may be optionally fused to aryl rings (including benzene rings) optionally having one or more substituents, carbocycle rings or heterocyclic rings. Examples of “aryl” groups include, but are not limited to, phenyl, naphthyl, tetrahydronaphthyl, biphenyl, indanyl, anthracyl or phenanthryl, as well as substituted derivatives thereof.

As used herein, the term “heteroaryl” refers to an optionally substituted monocyclic five to six-membered aromatic ring containing one or more heteroatomic substitutions selected from S, SO, SO₂, O, N, or N-oxide, or refers to such an aromatic ring fused to one or more rings such as heteroaryl rings, aryl rings, heterocyclic rings, or carbocycle rings (e.g., a bicyclic or tricyclic ring system), each having optional substituents.

Examples of optional substituents are selected from the group consisting of substituted C₁₋₃ alkyl, substituted C₂₋₃ alkenyl, substituted C₂₋₃ alkynyl, heteroaryl, heterocyclic, aryl, C₁₋₃ alkoxy optionally having one to three fluorine substituents, aryloxy, aralkoxy, acyl, aroyl, heteroaroyl, acyloxy, aroyloxy, heteroaroyloxy, sulfanyl, sulfinyl, sulfonyl, aminosulfonyl, sulfonylamino, carboxyamide, aminocarbonyl, carboxy, oxo, hydroxy, mercapto, amino, nitro, cyano, halogen or ureido. Examples of “heteroaryl” groups used herein include, but are not limited to, benzoimidazolyl, benzothiazolyl, benzoisothiazolyl, benzothiophenyl, benzopyrazinyl, benzotriazolyl, benzo[1,4]dioxanyl, benzofuranyl, 9H-a-carbolinyl, cinnolinyl, furanyl, furo[2,3-b]pyridinyl, imidazolyl, imidazolidinyl, imidazopyridinyl, isoxazolyl, isothiazolyl, isoquinolinyl, indolyl, indazolyl, indolizinyl, naphthyridinyl, oxazolyl, oxothiadiazolyl, oxadiazolyl, phthalazinyl, pyridyl, pyrrolyl, purinyl, pteridinyl, phenazinyl, pyrazolyl, pyridyl, pyrazolopyrimidinyl, pyrrolizinyl, pyridazyl, pyrazinyl, pyrimidyl, 4-oxo-1,2-dihydro-4H-pyrrolo[3,2,1-ij]-quinolin-4-yl, quinoxalinyl, quinazolinyl, quinolinyl, quinolizinyl, thiophenyl, triazolyl, triazinyl, tetrazolopyrimidinyl, triazolopyrimidinyl, tetrazolyl, thiazolyl, thiazolidinyl, and substituted versions thereof.

As used herein, the term “heterocyclic” refers to a three to seven-membered ring containing one or more heteroatomic moieties selected from S, SO, SO₂, O, N, or N-oxide, optionally substituted with one or more substituents selected from the group which includes substituted C₁₋₃ alkyl, substituted C₂₋₃ alkenyl, substituted C₂₋₃ alkynyl, heteroaryl, heterocyclic, aryl, C₁₋₃ alkoxy optionally having one to three fluorine substituents, aryloxy, aralkoxy, acyl, aroyl, heteroaroyl, acyloxy, aroyloxy, heteroaroyloxy, sulfanyl, sulfinyl, sulfonyl, aminosulfonyl, sulfonylamino, carboxyamide, aminocarbonyl, carboxy, oxo, hydroxy, mercapto, amino, nitro, cyano, halogen, and ureido. Such a ring can be saturated or have one or more degrees of unsaturation. Such a ring may be optionally fused to one or more “heterocyclic” ring(s), aryl ring(s), heteroaryl ring(s) or carbocycle ring(s), each having optional substituents.

Examples of “heterocyclic” moieties include, but are not limited to, 1,4-dioxanyl, 1,3-dioxanyl, pyrrolidinyl, pyrrolidin-2-onyl, piperidinyl, imidazolidine-2,4-dionepiperidinyl, piperazinyl, piperazine-2,5-dionyl, morpholinyl, dihydropyranyl, dihydrocinnolinyl, 2,3-dihydrobenzo[1,4]dioxinyl, 3,4-dihydro-2H-benzo[b][1,4]-dioxepinyl, tetrahydropyranyl, 2,3-dihydrofuranyl, 2,3-dihydrobenzofuranyl, dihydroisoxazolyl, tetrahydrobenzodiazepinyl, tetrahydroquinolinyl, tetrahydrofuranyl, tetrahydronaphthyridinyl, tetrahydropurinyl, tetrahydrothiopyranyl, tetrahydrothiophenyl, tetrahydroquinoxalinyl, tetrahydropyridinyl, tetrahydrocarbolinyl, 4H-benzo[1,3]-dioxinyl, benzo[1,3]dioxonyl, 2,2-difluorobenzo-[1,3]-dioxonyl, 2,3-dihydro-phthalazine-1,4-dionyl, and isoindole-1,3-dionyl.

As used herein, the term “alkoxy” refers to the group —ORₐ, where Rₐ is alkyl as defined above. Exemplary alkoxy groups useful in the present invention include, but are not limited to, methoxy, difluoromethoxy, trifluoromethoxy, ethoxy, n-propoxy, isopropoxy, n-butoxy and t-butoxy.

As used herein the term “aralkoxy” refers to the group —ORₐR_{b}, wherein Rₐ is alkyl and R_{b} is aryl as defined above.

As used herein the term “aryloxy” refers to the group —OR_{b}, wherein R_{b} is aryl as defined above.

As used herein, the term “mercapto” refers to the group —SH.

As used herein, the term “sulfanyl” refers to the group —SR_{c}, wherein R_{c} is substituted alkyl, substituted carbocycle, aryl, heteroaryl or heterocyclic, as defined above.

As used herein, the term “sulfinyl” refers to the group —S—(O)R_{c}, wherein R_{c} is substituted alkyl, substituted carbocycle, aryl, heteroaryl or heterocyclic, as defined above.

As used herein, the term “sulfonyl” refers to the group —S(O)₂R_{c}, wherein R_{c} is substituted alkyl, substituted carbocycle, aryl, heteroaryl or heterocyclic, as defined above.

As used herein, the term “oxo” refers to the group ═O.

As used herein, the term “hydroxy” refers to the group —OH.

As used herein, the term “amino” refers to the group —NH₂. The amino group is optionally substituted by substituted alkyl, substituted carbocycle, aryl, heteroaryl or heterocyclic, as defined above.

As used herein, the term “cyano” refers to the group —CN.

As used herein, the term “aminosulfonyl” refers to the group —S(O)₂NH₂. The aminosulfonyl group is optionally substituted by substituted alkyl, substituted carbocycle, aryl, heteroaryl or heterocyclic, as defined above.

As used herein, the term “sulfonylamino” refers to the group —NHS(O)₂R_{c} wherein R_{c} is substituted alkyl, substituted carbocycle, aryl, heteroaryl or heterocyclic, as defined above.

As used herein, the term “carboxyamide” refers to the group —NHC(O)R_{c} wherein R_{c} is substituted alkyl, substituted carbocycle, aryl, heteroaryl or heterocyclic, as defined above.

As used herein, the term “carboxy” refers to the group —C(O)OH. The carboxy group is optionally substituted by substituted alkyl, substituted carbocycle, aryl, heteroaryl or heterocyclic, as defined above.

As used herein, the term “aminocarbonyl” refers to the group —C(O)NH₂. The aminocarbonyl group is optionally substituted by substituted alkyl, substituted carbocycle, aryl, heteroaryl or heterocyclic, as defined above.

As used herein, the term “ureido” refers to the group —NHC(O)NHR_{d} wherein R_{d} is hydrogen, alkyl, carbocycle or aryl as defined above.

As used herein, the term “guanidino” refers to the group —NHC(═NH)NH₂.

As used herein, the term “acyl” refers to the group —C(O)Rₑ, wherein Rₑ is alkyl, carbocycle, or heterocyclic as defined herein.

As used herein, the term “aroyl” refers to the group —C(O)R_{b}, wherein R_{b} is aryl as defined herein.

As used herein, the term “heteroaroyl” refers to the group —C(O)R_{f}, wherein R_{f} is heteroaryl as defined herein.

As used herein, the term “acyloxy” refers to the group —OC(O)Rₑ, wherein Rₑ is alkyl, carbocycle, or heterocyclic as defined herein.

As used herein, the term “aroyloxy” refers to the group —OC(O)R_{b}, wherein R_{b} is aryl as defined herein.

As used herein, the term “heteroaroyloxy” refers to the group —OC(O)R_{f}, wherein R_{f} is heteroaryl as defined herein.

It is to be understood that the present invention also includes a pharmaceutically acceptable salt and an addition salt of the inventive compound, such as a hydrochloride, hydrobromide or trifluoroacetate addition salt and a sodium, potassium and magnesium salt.

The compounds of the present invention may contain one or more asymmetric carbon atoms and may exist in racemic and optically active forms. All of these compounds and diastereomers are incorporated within the scope of the present invention.

One embodiment of the present invention is to provide a compound of formula (Ia) or a pharmaceutically acceptable salt thereof:

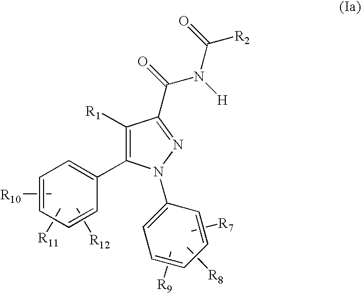

wherein, R₁, R₂, R₇, R₈, R₉, R₁₀, R₁₁ and R₁₂ have the same meanings as defined above.

Another embodiment of the present invention is to provide a compound of formula (Ib) or a pharmaceutically acceptable salt thereof:

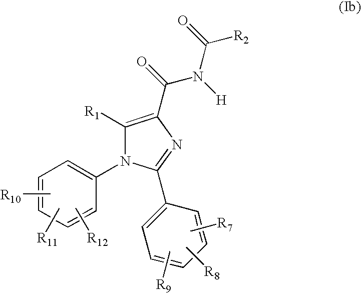

wherein, R₁, R₂, R₇, R₈, R₉, R₁₀, R₁₁ and R₁₂ have the same meanings as defined above.

A still another embodiment of the present invention is to provide a compound of formula (Ic) or a pharmaceutically acceptable salt thereof:

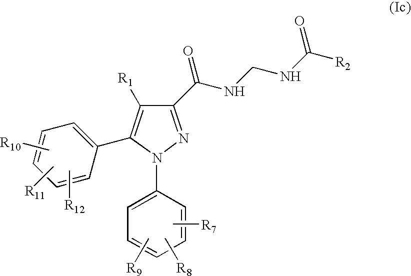

wherein, R₁, R₂, R₇, R₈, R₉, R₁₀, R₁₁, and R₁₂ have the same meanings as defined above.

Among the compound of the formula (Ic), preferred are those wherein: R₂ is hydrogen, optionally substituted C₁₋₇ alkyl, optionally substituted C₂₋₇ alkenyl, optionally substituted C₂₋₇ alkynyl, optionally substituted C₃₋₇ cycloalkyl, optionally substituted aryl, optionally substituted heteroaryl, or optionally substituted heterocycloalkyl.

Compounds useful in the present invention are selected from the group consisting of:
5-(4-Chlorophenyl)-1-(2,4-dichlorophenyl)-N-(2-ethylbutanoyl)-4-methyl-1H-pyrazole-3-carboxamide;
5-(4-Chlorophenyl)-N-(cyclopropanecarbonyl)-1-(2,4-dichlorophenyl)-4-methyl-1H-pyrazole-3-carboxamide;
5-(4-Chlorophenyl)-N-(cyclobutanecarbonyl)-1-(2,4-dichlorophenyl)-4-methyl-1H-pyrazole-3-carboxamide;
5-(4-Chlorophenyl)-N-(cyclopentanecarbonyl)-1-(2,4-dichlorophenyl)-4-methyl-1H-pyrazole-3-carboxamide;
5-(4-Chlorophenyl)-N-(cyclohexanecarbonyl)-1-(2,4-dichlorophenyl)-4-methyl-1H-pyrazole-3-carboxamide;
N-Acetyl-5-(4-chlorophenyl)-1-(2,4-dichlorophenyl)-4-methyl-1H-pyrazole-3-carboxamide;
N-Butyryl-5-(4-chlorophenyl)-1-(2,4-dichlorophenyl)-4-methyl-1H-pyrazole-3-carboxamide;
5-(4-Chlorophenyl)-1-(2,4-dichlorophenyl)-N-hexanoyl-4-methyl-1H-pyrazole-3-carboxamide;
5-(4-Chlorophenyl)-1-(2,4-dichlorophenyl)-N-isobutyryl-4-methyl-1H-pyrazole-3-carboxamide;
5-(4-Chlorophenyl)-1-(2,4-dichlorophenyl)-4-methyl-N-pivaloyl-1H-pyrazole-3-carboxamide;
5-(4-Chlorophenyl)-1-(2,4-dichlorophenyl)-N-(2,2-dimethylbutanoyl)-4-methyl-1H-pyrazole-3-carboxamide;
5-(4-Chlorophenyl)-1-(2,4-dichlorophenyl)-N-(3,3-dimethylbutanoyl)-4-methyl-1H-pyrazole-3-carboxamide;
5-(4-Chlorophenyl)-1-(2,4-dichlorophenyl)-4-methyl-N-(2-propylpentanoyl)-1H-pyrazole-3-carboxamide;
5-(4-Chlorophenyl)-1-(2,4-dichlorophenyl)-N-(2-ethylhexanoyl)-4-methyl-1H-pyrazole-3-carboxamide;
5-(4-Chlorophenyl)-1-(2,4-dichlorophenyl)-N-(hexylcarbamoyl)-4-methyl-1H-pyrazole-3-carboxamide;
5-(4-Chlorophenyl)-N-(cyclohexylcarbamoyl)-1-(2,4-dichlorophenyl)-4-methyl-1H-pyrazole-3-carboxamide;
5-(4-Chlorophenyl)-N-(cycloheptylcarbamoyl)-1-(2,4-dichlorophenyl)-4-methyl-1H-pyrazole-3-carboxamide;
5-(4-Chlorophenyl)-N-(cyclohexylmethylcarbamoyl)-1-(2,4-dichlorophenyl)-4-methyl-1H-pyrazole-3-carboxamide;
5-(4-Chlorophenyl)-1-(2,4-dichlorophenyl)-N-(isopropylcarbamoyl)-4-methyl-1H-pyrazole-3-carboxamide;
N-(tert-butylcarbamoyl)-5-(4-chlorophenyl)-1-(2,4-dichlorophenyl)-4-methyl-1H-pyrazole-3-carboxamide;
5-(4-Chlorophenyl)-1-(2,4-dichlorophenyl)-4-methyl-N-(phenylcarbamoyl)-1H-pyrazole-3-carboxamide;
N-(Benzylcarbamoyl)-5-(4-chlorophenyl)-1-(2,4-dichlorophenyl)-4-methyl-1H-pyrazole-3-carboxamide;
Benzyl 5-(4-chlorophenyl)-1-(2,4-dichlorophenyl)-4-methyl-1H-pyrazole-3-carbonylcarbamate;
Butyl 5-(4-chlorophenyl)-1-(2,4-dichlorophenyl)-4-methyl-1H-pyrazole-3-carbonylcarbamate;
tert-Butyl 5-(4-chlorophenyl)-1-(2,4-dichlorophenyl)-4-methyl-1H-pyrazole-3-carbonylcarbamate;
Neopentyl 5-(4-chlorophenyl)-1-(2,4-dichlorophenyl)-4-methyl-1H-pyrazole-3-carbonylcarbamate;
1-(4-Chlorophenyl)-2-(2,4-dichlorophenyl)-N-(2-ethylbutanoyl)-5-methyl-1H-imidazole-4-carboxamide;
1-(4-Chlorophenyl)-N-(cyclohexanecarbonyl)-2-(2,4-dichlorophenyl)-5-methyl-1H-imidazole-4-carboxamide;
1-(4-Chlorophenyl)-2-(2,4-dichlorophenyl)-5-methyl-N-pivaloyl-1H-imidazole-4-carboxamide;
1-(4-Chlorophenyl)-2-(2,4-dichlorophenyl)-N-(2,2-dimethylbutanoyl)-5-methyl-1H-imidazole-4-carboxamide;
1-(4-Chlorophenyl)-2-(2,4-dichlorophenyl)-5-methyl-N-(2-propylpentanoyl)-1H-imidazole-4-carboxamide;
1-(4-Chlorophenyl)-2-(2,4-dichlorophenyl)-N-(2-ethylhexanoyl)-5-methyl-1H-imidazole-4-carboxamide;
5-(4-Chlorophenyl)-1-(2,4-dichlorophenyl)-4-methyl-N-(octanamidomethyl)-1H-pyrazole-3-carboxamide;
5-(4-Chlorophenyl)-N-(cyclobutanecarboxamidomethyl)-1-(2,4-dichlorophenyl)-4-methyl-1H-pyrazole-3-carboxamide;
5-(4-Chlorophenyl)-N-(cyclohexanecarboxamidomethyl)-1-(2,4-dichlorophenyl)-4-methyl-1H-pyrazole-3-carboxamide;
N-(Butyramidomethyl)-5-(4-chlorophenyl)-1-(2,4-dichlorophenyl)-4-methyl-1H-pyrazole-3-carboxamide;
5-(4-Chlorophenyl)-1-(2,4-dichlorophenyl)-N-(isobutyramidomethyl)-4-methyl-1H-pyrazole-3-carboxamide;
5-(4-Chlorophenyl)-N-(cycloheptanecarboxamidomethyl)-1-(2,4-dichlorophenyl)-4-methyl-1H-pyrazole-3-carboxamide;
5-(4-Chlorophenyl)-N-((2-cyclohexylacetamido)methyl)-1-(2,4-dichlorophenyl)-4-methyl-1H-pyrazole-3-carboxamide;
5-(4-Chlorophenyl)-1-(2,4-dichlorophenyl)-4-methyl-N-((3-methylcyclohexanecarboxamido)methyl)-1H-pyrazole-3-carboxamide; and
5-(4-Chlorophenyl)-N-((2-cyclopentylacetamido)methyl)-1-(2,4-dichlorophenyl)-4-methyl-1H-pyrazole-3-carboxamide.

### General Synthetic Sequence

The following synthetic schemes are merely illustrative of the methods by which the compounds of the invention may be prepared and are not intended to limit the scope of the invention as defined in the appended claims.

### Synthesis of 1,5-diaryl-N-acyl pyrazole-3-carboxamide derivatives (compound of formula (Ia-1))

As shown in Reaction Scheme 1, the compound of formula (Ia-1) may be prepared by (i) converting a carboxylic acid derivative of formula (5) by conventional methods to a carbonyl chloride compound of formula (6); (ii) subjecting the carbonyl chloride compound of formula (6) to amination with an ammonium hydroxide to obtain an amide compound of formula (7); and (ii) coupling the amide compound of formula (7) with an acyl chloride compound of formula (8) in the presence of a base such as NaHMDS (sodium salt of hexamethyldisilazane) or sodium hydride at low temperature (e.g. −78° C. to room temperature) (See Weiguo, Liu. et al., Bioorg. Med. Chem. Lett. 2005, 15, 4574-4578):

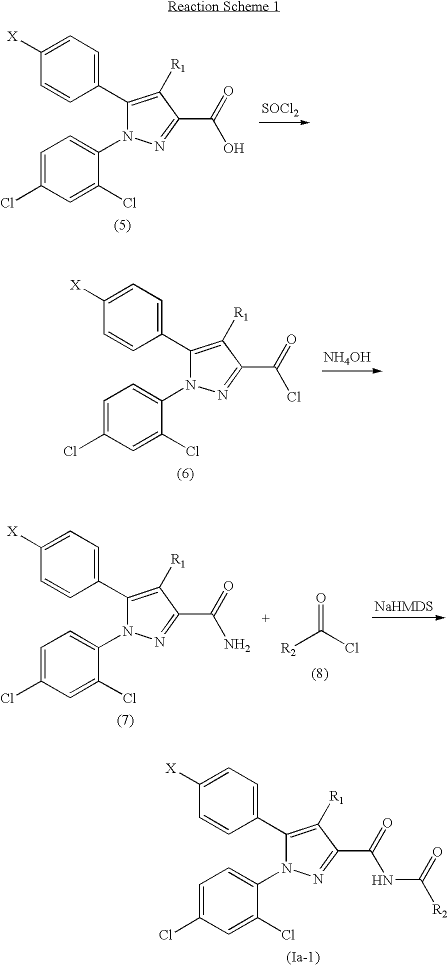

wherein R₁, and R₂ have the same meanings as defined above; and X is halogen.

### Preparation of 1,5-diaryl pyrazole-3-carboxylic acid as a synthetic intermediate

The carboxylic acid derivative of formula (5) used as a starting material in preparing the compound of formula (Ia-1) may be prepared by a conventional method, e.g., by treating an acetophenone derivative of formula (1) with an organic base such as lithium hexamethyldisilazide (LHMDS) to produce a corresponding alkali metal salt of formula (2), reacting the resulting salt with an equimolar amount of diethyl oxalate to provide a ketoester salt of formula (3), reacting the salt of formula (3) with a hydrazine derivative in refluxing acetic acid to obtain a pyrazole-3-carboxylic ester of formula (4), and transforming the ester of formula (4) into an acid form of formula (5) using an alkaline agent such as potassium hydroxide or lithium hydroxide, followed by acidification (See Barth, F. et al., U.S. Pat. No. 5,462,960), as shown in Reaction Scheme 2:

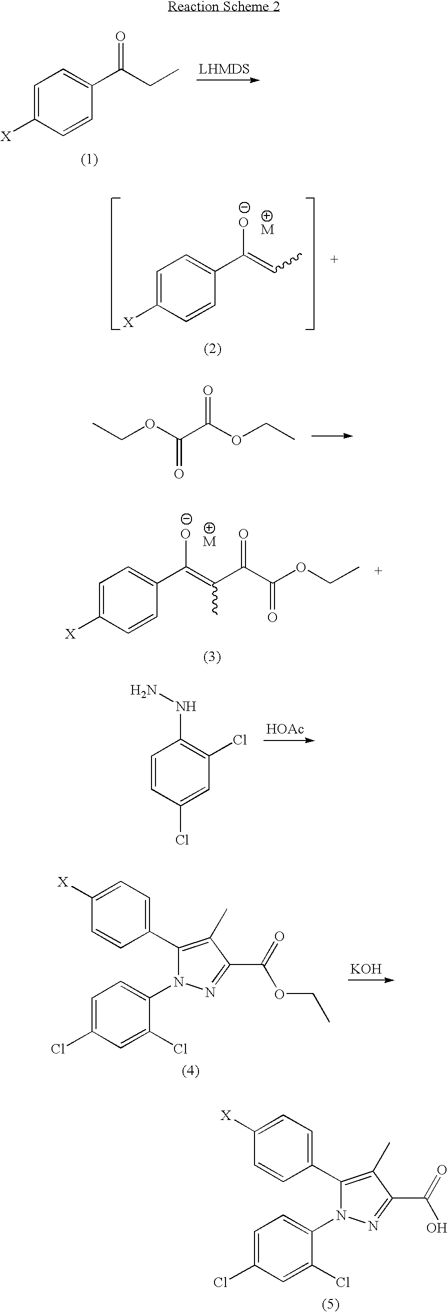

wherein X is halogen.

### Synthesis of 1,2-diaryl-N-acyl imidazole-4-carboxamide derivatives (compound of formula (Ib-1))

As shown in Reaction Scheme 3, the compound of formula (Ib-1) can be prepared by (i) converting a carboxylic acid derivative of formula (15) by conventional methods to a carbonyl chloride compound of formula (16); (ii) subjecting the carbonyl chloride compound of formula (16) to amination with an ammonium hydroxide to obtain an amide compound of formula (17); and (iii) coupling the amide compound of formula (17) with an acyl chloride compound of formula (8) in the presence of a base such as NaHMDS (sodium salt of hexamethyldisilazane) or sodium hydride at low temperature (e.g. −78° C. to room temperature) (See Weiguo, Liu. et al., Bioorg. Med. Chem. Lett. 2005, 15, 4574-4578):

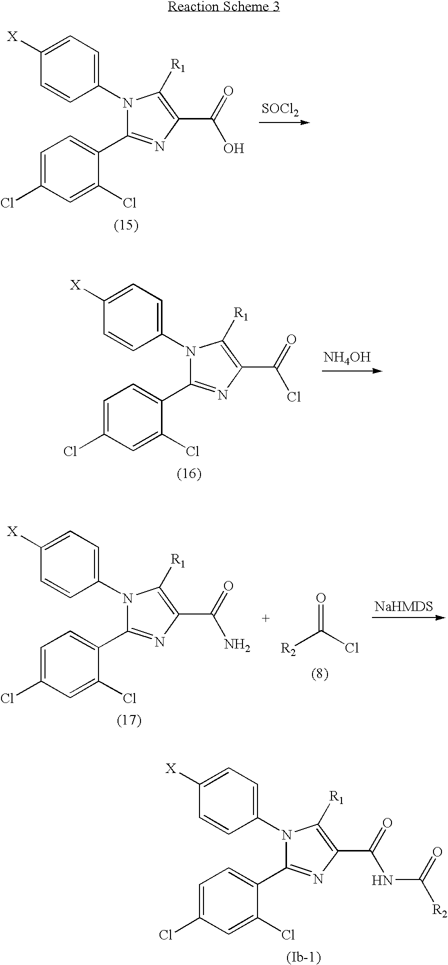

wherein R₁, and R₂ have the same meanings as defined above; and X is halogen.

### Preparation of 1,2-diaryl imidazole-4-carboxylic acid as a synthetic intermediate

As shown in Reaction Scheme 4, The carboxylic acid derivative of formula (15) used as starting material in preparing the compound of formula (Ib-1) may be prepared by a conventional method, e.g., by reacting a benzonitrile derivative of formula (10) with an aniline derivative of formula (11) such as 4-chloroaniline using a non-nucleophilic base such as sodium bis(trimethylsilyl)amide (NaHMDS) to produce a corresponding arylbenzamidine of formula (12), subsequently reacting the resulting arylbenzamidine of formula (12) with ethyl 3-bromo-2-oxobutanoate of formula (13) to provide an intermediate ethyl 1,2-diaryl-5-methyl-1H-imidazole-4-carboxylate of formula (14), then transforming the intermediate of formula (14) into an acid form of formula (15) using an alkaline agent such as potassium hydroxide or lithium hydroxide, followed by acidification (See Lange, J. H. M. et al., J. Med. Chem. 2005, 48, 1823):

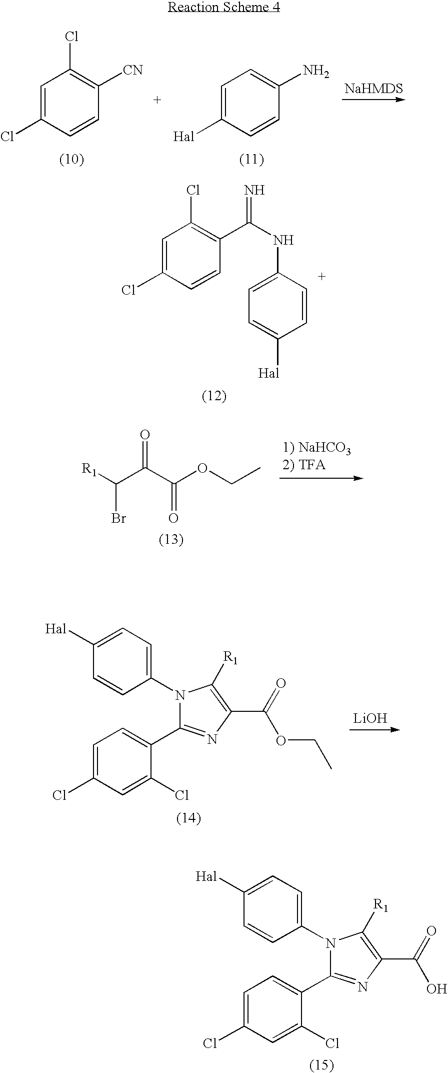

wherein R₁ has the same meanings as defined above.

### Synthesis of N-acylaminomethyl-1,5-diaryl pyrazole-3-carboxamide derivatives (compound of formula (Ic-1))

As shown in Reaction Scheme 5, a compound of formula (Ic-1) may be prepared by (i) coupling a carboxylic acid derivative of formula (5) with glycinamide hydrochloride in the presence of a coupling agent such as EDCI/HOBt/NMM, to obtain a N-carbamoylmethyl amide compound of formula (19); (ii) reacting the N-carbamoylmethyl amide compound of formula (19) with [bis(trifluoroacetoxy)iodo]-benzene (PIFA) or iodine in a solvent such as a mixture of ACN and water, followed by treatment with 1 M hydrochloric acid to obtain the N-aminomethyl amide hydrochloric acid salt of formula (20); and (iii) coupling a N-aminomethyl amide hydrochloric acid salt of formula (20) with a compound of formula (21) in the presence of a coupling agent such as DMAP and EDCI:

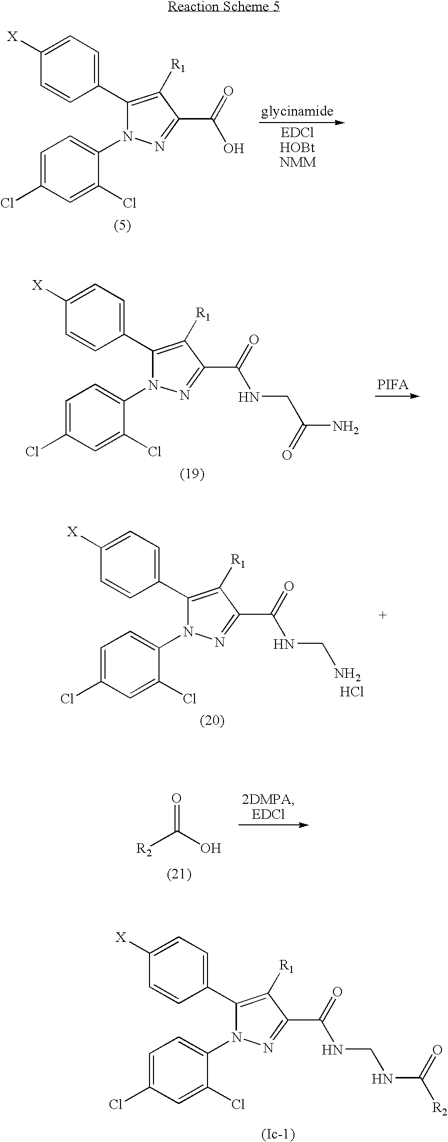

wherein R₁, and R₂ have the same meanings as defined above; and X is halogen.

### Binding Affinity for Selected CB1 Receptor Antagonists

| | | | IC₅₀ |
|---|---|---|---|
| No. | Structure | Name | (nM) |
| 1 | 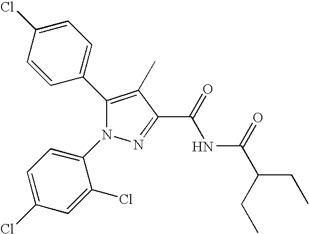 | 5-(4-chlorophenyl)-1-(2,4-dichlorophenyl)-N-(2-ethylbutanoyl)-4-methyl-1H-pyrazole-3-carboxamide | 24.8 |
| 2 | 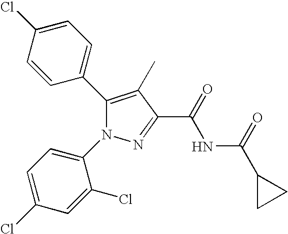 | 5-(4-chlorophenyl)-N-(cyclopropanecarbonyl)-1-(2,4-dichlorophenyl)-4-methyl-1H-pyrazole-3-carboxamide | 124 |
| 3 | 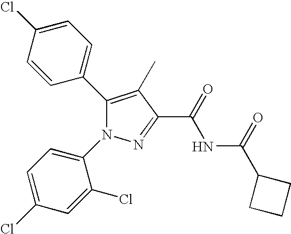 | 5-(4-chlorophenyl)-N-(cyclobutanecarbonyl)-1-(2,4-dichlorophenyl)-4-methyl-1H-pyrazole-3-carboxamide | 127 |
| 4 | 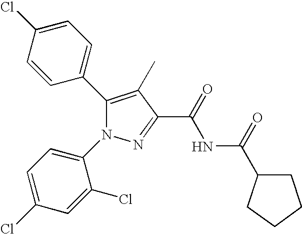 | 5-(4-chlorophenyl)-N-(cyclopentanecarbonyl)-1-(2,4-dichlorophenyl)-4-methyl-1H-pyrazole-3-carboxamide | 67.2 |
| 5 | 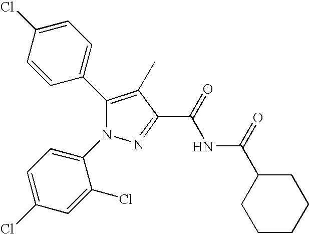 | 5-(4-chlorophenyl)-N-(cyclohexanecarbonyl)-1-(2,4-dichlorophenyl)-4-methyl-1H-pyrazole-3-carboxamide | 30.4 |
| 6 | 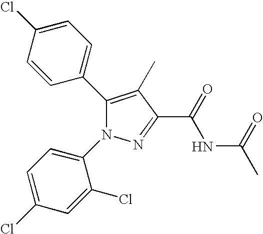 | N-acetyl-5-(4-chlorophenyl)-1-(2,4-dichlorophenyl)-4-methyl-1H-pyrazole-3-carboxamide | 383 |
| 7 | 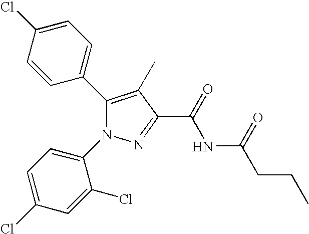 | N-butyryl-5-(4-chlorophenyl)-1-(2,4-dichlorophenyl)-4-methyl-1H-pyrazole-3-carboxamide | 43.7 |
| 8 | 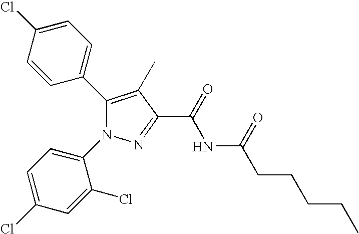 | 5-(4-chlorophenyl)-1-(2,4-dichlorophenyl)-N-hexanoyl-4-methyl-1H-pyrazole-3-carboxamide | 33.5 |
| 9 | 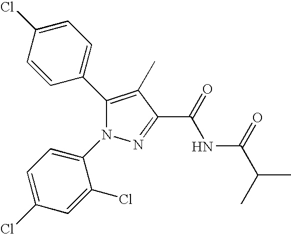 | 5-(4-chlorophenyl)-1-(2,4-dichlorophenyl)-N-isobutyryl-4-methyl-1H-pyrazole-3-carboxamide | 161 |
| 10 | 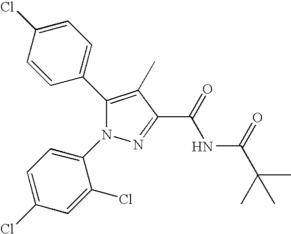 | 5-(4-chlorophenyl)-1-(2,4-dichlorophenyl)-4-methyl-N-pivaloyl-1H-pyrazole-3-carboxamide | 32.7 |
| 11 | 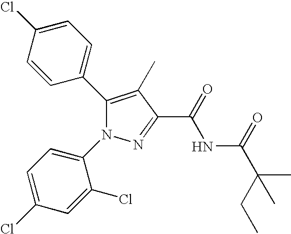 | 5-(4-chlorophenyl)-1-(2,4-dichlorophenyl)-N-(2,2-dimethylbutanoyl)-4-methyl-1H-pyrazole-3-carboxamide | 21.2 |
| 12 | 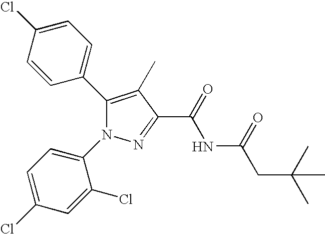 | 5-(4-chlorophenyl)-1-(2,4-dichlorophenyl)-N-(3,3-dimethylbutanoyl)-4-methyl-1H-pyrazole-3-carboxamide | 53.4 |
| 13 | 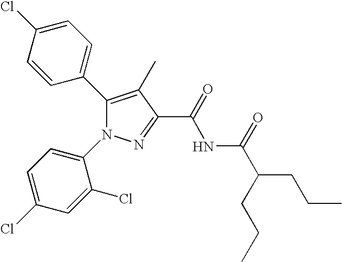 | 5-(4-chlorophenyl)-1-(2,4-dichlorophenyl)-4-methyl-N-(2-propylpentanoyl)-1H-pyrazole-3-carboxamide | 26.9 |
| 14 | 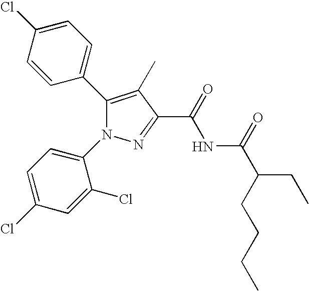 | 5-(4-chlorophenyl)-1-(2,4-dichlorophenyl)-N-(2-ethylhexanoyl)-4-methyl-1H-pyrazole-3-carboxamide | 43.1 |
| 15 | 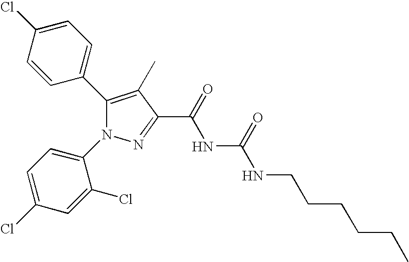 | 5-(4-chlorophenyl)-1-(2,4-dichlorophenyl)-N-hexylcarbamoyl)-4-methyl-1H-pyrazole-3-carboxamide | 138 |
| 16 | 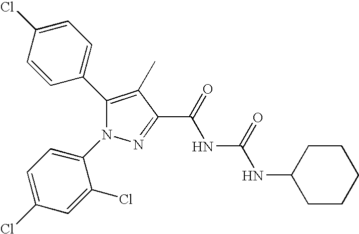 | 5-(4-chlorophenyl)-N-(cyclohexylcarbamoyl)-1-(2,4-dichlorophenyl)-4-methyl-1H-pyrazole-3-carboxamide | 390 |
| 17 | 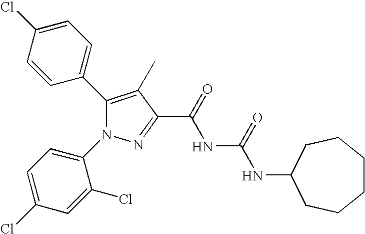 | 5-(4-chlorophenyl)-N-(cycloheptylcarbamoyl)-1-(2,4-dichlorophenyl)-4-methyl-1H-pyrazole-3-carboxamide | 750 |
| 18 | 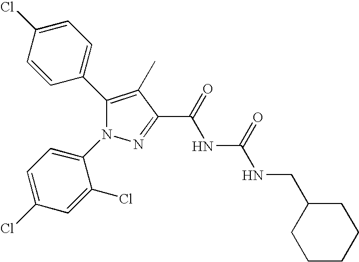 | 5-(4-chlorophenyl)-N-(cyclohexylmethylcarbamoyl)-1-(2,4-dichlorophenyl)-4-methyl-1H-pyrazole-3-carboxamide | insoluble |
| 19 | 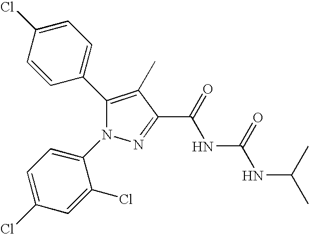 | 5-(4-chlorophenyl)-1-(2,4-dichlorophenyl)-N-(isopropylcarbamoyl)-4-methyl-1H-pyrazole-3-carboxamide | 253 |
| 20 | 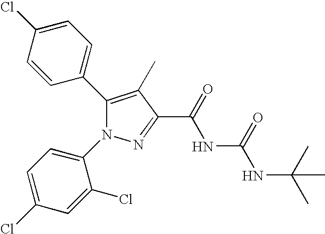 | N-(tert-butylcarbamoyl)-5-(4-chlorophenyl)-1-(2,4-dichlorophenyl)-4-methyl-1H-pyrazole-3-carboxamide | insoluble |
| 21 | 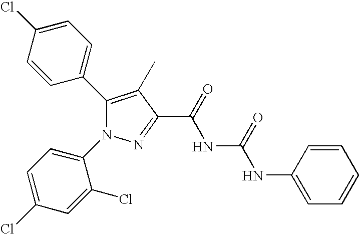 | 5-(4-chlorophenyl)-1-(2,4-dichlorophenyl)-4-methyl-N-(phenylcarbamoyl)-1H-pyrazole-3-carboxamide | >10 um |
| 22 | 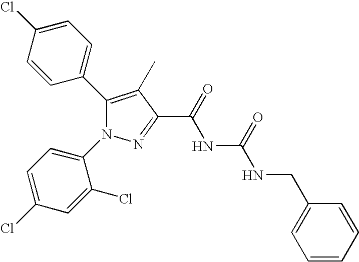 | N-(benzylcarbamoyl)-5-(4-chlorophenyl)-1-(2,4-dichlorophenyl)-4-methyl-1H-pyrazole-3-carboxamide | 146 |
| 23 | 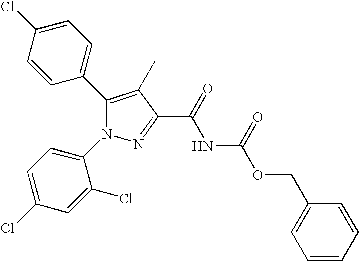 | benzyl5-(4-chlorophenyl)-1-(2,4-dichlorophenyl)-4-methyl-1H-pyrazole-3-carbonylcarbamate | 17.8 |
| 24 | 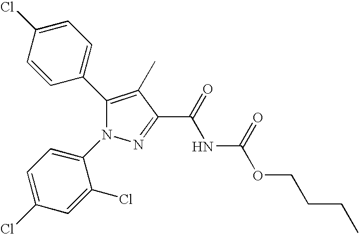 | butyl5-(4-chlorophenyl)-1-(2,4-dichlorophenyl)-4-methyl-1H-pyrazole-3-carbonylcarbamate | 99.7 |
| 25 | 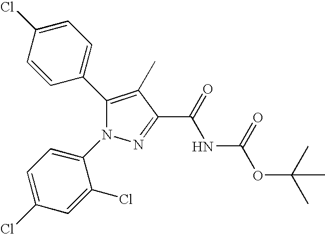 | tert-butyl5-(4-chlorophenyl)-1-(2,4-dichlorophenyl)-4-methyl-1H-pyrazole-3-carbonylcarbamate | 82.5 |
| 26 | 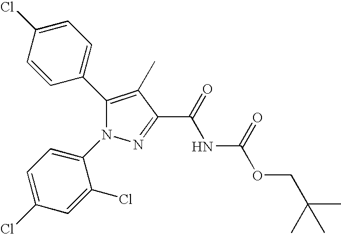 | neopentyl5-(4-chlorophenyl)-1-(2,4-dichlorophenyl)-4-methyl-1H-pyrazole-3-carbonylcarbamate | 363 |
| 27 | 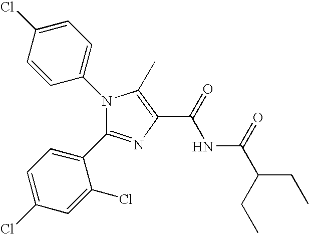 | 1-(4-chlorophenyl)-2-(2,4-dichlorophenyl)-N-(2-ethylbutanoyl)-5-methyl-1H-imidazole-4-carboxamide | 20.0 |
| 28 | 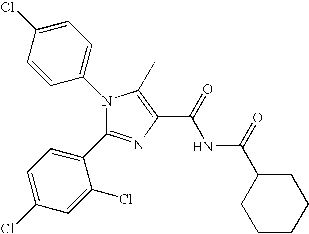 | 1-(4-chlorophenyl)-N-(cyclohexanecarbonyl)-2-(2,4-dichlorophenyl)-5-methyl-1H-imidazole-4-carboxamide | 88.0 |
| 29 | 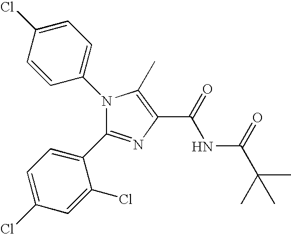 | 1-(4-chlorophenyl)-2-(2,4-dichlorophenyl)-5-methyl-N-pivaloyl-1H-imidazole-4-carboxamide | 40.0 |
| 30 | 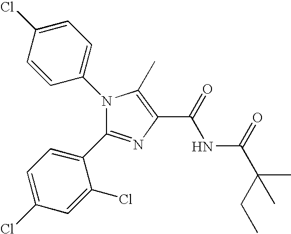 | 1-(4-chlorophenyl)-2-(2,4-dichlorophenyl)-N-(2,2-dimethylbutanoyl)-5-methyl-1H-imidazole-4-carboxamide | 39.9 |
| 31 | 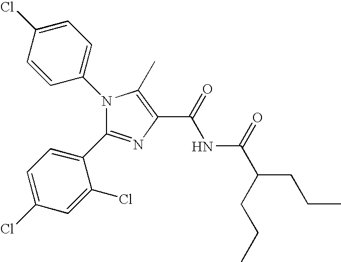 | 1-(4-chlorophenyl)-2-(2,4-dichlorophenyl)-5-methyl-N-(2-propylpentanoyl)-1H-imidazole-4-carboxamide | 75.4 |
| 32 | 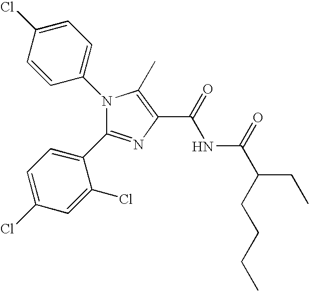 | 1-(4-chlorophenyl)-2-(2,4-dichlorophenyl)-N-(2-ethylhexanoyl)-5-methyl-1H-imidazole-4-carboxamide | 46.6 |
| 33 | 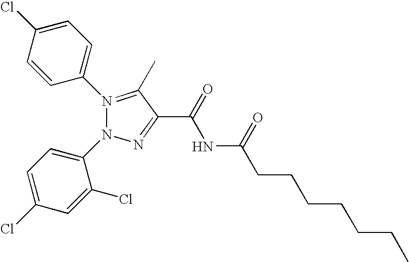 | 5-(4-chlorophenyl)-1-(2,4-dichlorophenyl)-4-methyl-N-(octanamidomethyl)-1H-pyrazole-3-carboxamide | 1180 |
| 34 | 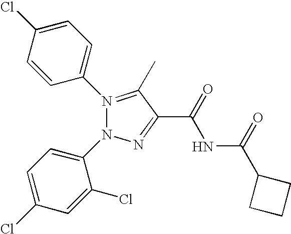 | 5-(4-chlorophenyl)-N-(cyclobutanecarboxamidomethyl)-1-(2,4-dichlorophenyl)-4-methyl-1H-pyrazole-3-carboxamide | 150 |
| 35 | 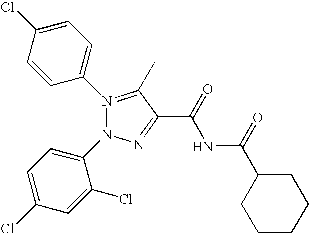 | 5-(4-chlorophenyl)-N-(cyclohexanecarboxamidomethyl)-1-(2,4-dichlorophenyl)-4-methyl-1H-pyrazole-3-carboxamide | 49.2 |
| 36 | 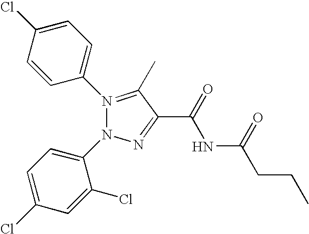 | N-(butyramidomethyl)-5-(4-chlorophenyl)-1-(2,4-dichlorophenyl)-4-methyl-1H-pyrazole-3-carboxamide | 905 |
| 37 | 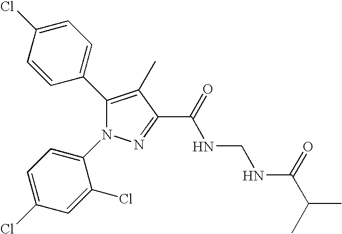 | 5-(4-chlorophenyl)-1-(2,4-dichlorophenyl)-N-(isobutyramidomethyl)-4-methyl-1H-pyrazole-3-carboxamide | 951 |
| 38 | 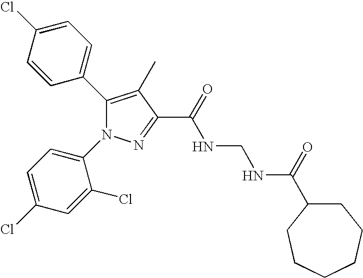 | 5-(4-chlorophenyl)-N-(cycloheptanecarboxamidomethyl)-1-(2,4-dichlorophenyl)-4-methyl-1H-pyrazole-3-carboxamide | 75.6 |
| 39 | 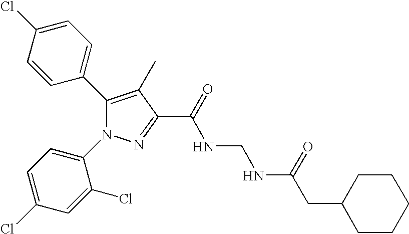 | 5-(4-chlorophenyl)-N-((2-cyclohexylacetamido)methyl)-1-(2,4-dichlorophenyl)-4-methyl-1H-pyrazole-3-carboxamide | 46.5 |
| 40 | 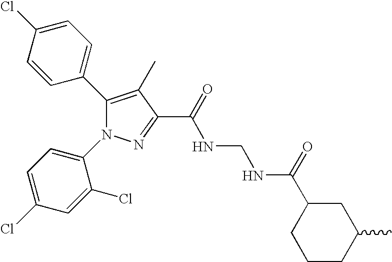 | 5-(4-chlorophenyl)-1-(2,4-dichlorophenyl)-4-methyl-N-((3-methylcyclohexanecarboxamido)methyl)-1H-pyrazole-3-carboxamide | 102 |
| 41 | 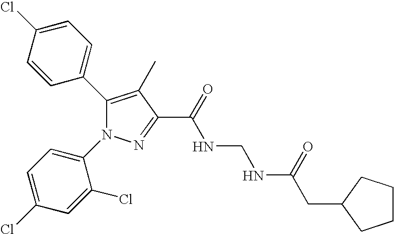 | 5-(4-chlorophenyl)-N-((2-cyclopentylacetamido)methyl)-1-(2,4-dichlorophenyl)-4-methyl-1H-pyrazole-3-carboxamide | 138 |

The inventive azole compound of formula (I) is effective as a cannabinoid CB₁ receptor inverse agonist or antagonist, thereby preventing or treating obesity and obesity-related metabolic disorders.

Accordingly, the present invention provides a pharmaceutical composition for preventing or treating obesity and obesity-related metabolic disorders, which comprises the compound of formula (I) or a pharmaceutically acceptable salt thereof as an active ingredient and a pharmaceutically acceptable carrier.

Further, the present invention provides a method for preventing or treating obesity and obesity-related metabolic disorders in a mammal, which comprises administering the compound of formula (I) of the present invention to the mammal.

Also, the present invention provides a method for inhibiting cannabinoid CB₁ receptor in a mammal, which comprises administering the compound of formula (I) of the present invention to the mammal.

As used herein, the term “obesity-related metabolic disorders” refers to chronic diseases that require treatment to reduce the excessive health risks associated with obesity and exemplary disorders include type 2 diabetes mellitus, cardiovascular and hypertension, hyperlipidaemia, fibrinolytic abnormalities.

The pharmaceutical composition may be administered orally, intramuscularly or subcutaneously. The formulation for oral administration may take various forms such as a syrup, tablet, capsule, cream and lozenge. A syrup formulation will generally contain a suspension or solution of the compound or its salt in a liquid carrier, e.g., ethanol, peanut oil, olive oil, glycerine or water, optionally with a flavoring or coloring agent. When the composition is in the form of a tablet, any one of pharmaceutical carriers routinely used for preparing solid formulations may be used. Examples of such carriers include magnesium stearate, terra alba, talc, gelatin, acacia, stearic acid, starch, lactose and sucrose. When the composition is in the form of a capsule, any of the routine encapsulation procedures may be employed, e.g., using the aforementioned carriers in a hard gelatin capsule shell. When the composition is formulated in the form of a soft gelatin shell capsule, any of the pharmaceutical carrier routinely used for preparing dispersions or suspensions may be prepared using an aqueous gum, cellulose, silicate or oil. The formulation for intramuscular or subcutaneous administration may take a liquid form such as a solution, suspension and emulsion which includes aqueous solvents such as water, physiological saline and Ringer's solution; or lipophilic solvents such as fatty oil, sesame oil, corn oil and synthetic fatty acid ester.

Preferably the composition is formulated in a specific dosage form for a particular patient.

Each dosage unit for oral administration contains suitably from 0.1 mg to 500 mg/Kg, and preferably from 1 mg to 100 mg/Kg of the compound of Formula (I) or its pharmaceutically acceptable salt.

The suitable daily dosage for oral administration is about 0.01 mg/Kg to 40 mg/Kg of the compound of Formula (I) or its pharmaceutically acceptable salt, may be administered 1 to 6 times a day, depending on the patient's condition.

### SYNTHETIC EXAMPLES

The invention will now be described by reference to the following examples which are merely illustrative and not to be construed as a limitation of the scope of the present invention.

As used herein the symbols and conventions used in these processes, schemes and examples are consistent with those used in the contemporary scientific literature, for example, the Journal of the American Chemical Society or the Journal of Biological Chemistry. Unless otherwise noted, all starting materials were obtained from commercial suppliers and used without further purification.

| | |
|---|---|
| Hz (Hertz); | TLC (thin layer chromatography); |
| Tᵣ (retention time); | RP (reverse phase); |
| MeOH (methanol); | i-PrOH (isopropanol); |
| TFA (trifluoroacetic acid); | TEA (triethylamine); |
| EtOH (ethanol); | THF (tetrahydrofuran); |
| DMSO (dimethylsulfoxide); | EtOAc (ethyl acetate); |
| DCM (dichlromethane); | DMF (N,N-dimethylformamide); |
| CDI (1,1-carbnyldiimidazole); | HOAc (acetic acid); |
| HOSu (N-hydroxysuccinimide); | Ac (acetyl); |
| HOBT (1-hydroxybenzotriazole); | |
| BOC (tert-butyloxycarbonyl); | |
| mCPBA (meta-chloroperbenzoic acid); | |
| FMOC (9-fluorenylmethoxycarbonyl); | |
| DCC (dicyclohexylcarbodiimide); | Cbz (benzyloxycarbonyl); |
| NMM (N-methyl morpholine); | |
| HOAt (1-hydroxy-7-azabenzotriazole); | |
| TBAF (tetra-n-butylammonium fluoride); | |
| THP (tetrahydro-2H-pyran-2-yl); | |
| DMAP (4-dimethylaminopyridine); | Bn (benzyl); |
| HPLC (high pressure liquid chromatography); | |
| rt (room temperature); | |
| BOP (bis(2-oxo-3-oxazolidinyl)phosphinic chloride); | |
| EDCI (1-ethyl-3-[3-dimethylaminopropyl]carbodiimide hydrochloride); | |
| HBTU (O-Benzotriazole1-yl-N,N,N′,N′-tetramethyluronium | |
| hexafluorophosphate); | |
| PIFA ([bis(trifluoroacetoxy)iodo]-benzene). | |

All references to ether are to diethyl ether; brine refers to a saturated aqueous solution of NaCl. Unless otherwise indicated, all temperatures are expressed in ° C. (degrees Centigrade). All reactions are conducted under an inert atmosphere at rt unless otherwise noted, and all solvents are highest available purity unless otherwise indicated.

¹H NMR spectra were recorded on either a Jeol ECX-400, or a Jeol JNM-LA300 spectrometer. Chemical shifts are expressed in parts per million (ppm, units). Coupling constants are in units of hertz (Hz). Splitting patterns describe apparent multiplicities and are designated as s (singlet), d (doublet), t (triplet), q (quartet), quint (quintet), m (multiplet), br (broad).

Mass spectra were run on either a Micromass, Quattro LC Triple Quadruple Tandem Mass Spectometer, ESI or Agilent, 1100LC/MSD, ESI.

For preparative HPLC, ca 100 mg of the final products were injected in 1 mL of DMSO onto a SunFire™ Prep C18 OBD 5 um 19×100 mm Column with a 10 min gradient from 10% CH₃CN to 90% CH₃CN in H₂O. Flash chromatography was run over Merck silica gel 60 (230-400 mesh). Most of the reactions were monitored by thin-layer chromatography on 0.25 mm E. Merck silica gel plates (60F-254), visualized with UV light, 5% ethanolic phosphomolybdic acid or p-anisaldehyde solution.

The following synthetic schemes are merely illustrative of the methods by which the compounds of the invention may be prepared and are not intended to limit the scope of the invention as defined in the appended claims.

The compounds disclosed in Example 2 to 14 were prepared following the general procedure described in Example 1.

The compounds disclosed in Example 16 to 22 were prepared following the general procedure described in Example 15.

The compounds disclosed in Example 24 to 26 were prepared following the general procedure described in Example 23.

The compounds disclosed in Example 28 to 32 were prepared following the general procedure described in Example 27.

The compounds disclosed in Example 34 to 41 were prepared following the general procedure described in Example 33.

### Preparation of 1,5-diaryl-N-acyl pyrazole-3-carboxamide

### Preparation 1

### 5-(4-Chlorophenyl)-1-(2,4-dichlorophenyl)-4-methyl-1H-pyrazole-3-carboxamide

To a suspension of 5-(4-chlorophenyl)-1-(2,4-dichlorophenyl)-4-methyl-1H-pyrazole-3-carboxylic acid (3.82 g, 10 mmol) in toluene (75 ml) was added thionyl chloride (3.64 ml, 50 mmol) and the mixture was refluxed for 3 hours and then cooled to the room temperature. The solvent was evaporated off under the reduced pressure. The residue was redissolved in toluene (30 ml) and the solvent was evaporated off again (procedure repeated twice) to yield the carboxyl chloride (3.94 g, 98% yield). Concentrated ammonium hydroxide solution (30 ml) was added dropwise to a solution of the carboxyl chloride obtained above in DCM (40 ml) at 0° C. The mixture was subsequently stirred at room temperature for 16 hours and then extracted with DCM (2×40 ml). The combined DCM was washed successively with water, dried over MgSO₄ and evaporated under vacuum to provide 3.56 g (9.3 mmol, 93% yield) of the title compound as a yellow solid.

¹H NMR (400 MHz, CDCl₃) δ 7.44 (d, J=2.0 Hz, 1H), 7.33-7.25 (m, 4H), 7.07 (d, J=8.4 Hz, 2H), 6.82 (br s, 1H, —NH—), 5.43 (br s, 1H, —NH—), 2.37 (s, 3H). MH+ 380.

### Example 1

### 5-(4-Chlorophenyl)-1-(2,4-dichlorophenyl)-N-(2-ethylbutanoyl)-4-methyl-1H-pyrazole-3-carboxamide

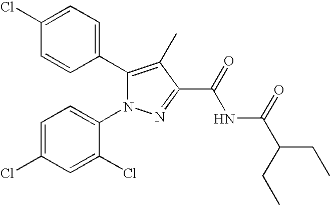

To a solution of 5-(4-chlorophenyl)-1-(2,4-dichlorophenyl)-4-methyl-1H-pyrazole-3-carboxamide (228 mg, 0.6 mmol) in THF (5 mL) was added 1M NaHMDS (0.9 mL, 0.9 mmol) at −78° C. under a nitrogen atmosphere. After stirring for 20 min, 2-ethylbutyryl chloride (80.8 mg, 0.6 mmol) dissolved in THF (1 mL) was added dropwise thereto, and the mixture was reacted for 30 min. Then, the mixture was returned to room temperature and further reacted for 16 hr. After completion of the reaction, the reaction mixture was pour into saturated NaHCO₃ solution (30 mL) and extracted with EtOAc (50 mL). The organic layer was washed successively with water, dried over MgSO₄ and evaporated under vacuum. The residue was further purified by prep HPLC to provide the title compound (111 mg, 0.23 mmol, 38%) as a pale yellow solid.

¹H NMR (400 MHz, CDCl₃) δ 9.37 (br s, 1H, —NH—), 7.45 (d, J=1.8 Hz, 1H), 7.34-7.24 (m, 4H), 7.06 (d, J=8.2 Hz, 2H), 3.33-3.26 (m, 1H), 2.38 (s, 3H), 1.84-1.73 (m, 2H), 1.66-1.55 (m, 2H), 0.97 (t, J=7.8 Hz, 6H). MH+ 478.

The following compounds of Examples 2 to 14 were obtained by repeating the procedure of Example 1.

### Example 2

### 5-(4-Chlorophenyl)-N-(cyclopropanecarbonyl)-1-(2,4-dichlorophenyl)-4-methyl-1H-pyrazole-3-carboxamide

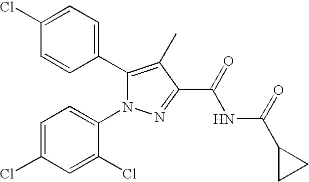

¹H NMR (400 MHz, CDCl₃) δ 9.42 (br s, 1H, —NH—), 7.44 (d, J=1.8 Hz, 1H), 7.34-7.24 (m, 4H), 7.07 (d, J=8.2 Hz, 2H), 3.04-2.97 (m, 1H), 2.39 (s, 3H), 1.23-1.19 (m, 2H), 1.05-1.00 (m, 2H). MH+ 448.

### Example 3

### 5-(4-Chlorophenyl)-N-(cyclobutanecarbonyl)-1-(2,4-dichlorophenyl)-4-methyl-1H-pyrazole-3-carboxamide

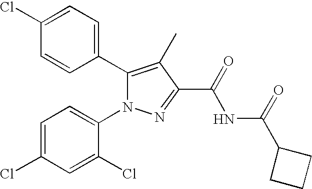

¹H NMR (400 MHz, CDCl₃) δ 9.28 (br s, 1H, —NH—), 7.44 (d, J=1.8 Hz, 1H), 7.34-7.24 (m, 4H), 7.06 (d, J=8.2 Hz, 2H), 4.06-3.97 (m, 1H), 2.44-2.28 (m, 7H), 2.09-1.99 (m, 1H), 1.97-1.86 (m, 1H). MH+ 462.

### Example 4

### 5-(4-Chlorophenyl)-N-(cyclopentanecarbonyl)-1-(2,4-dichlorophenyl)-4-methyl-1H-pyrazole-3-carboxamide

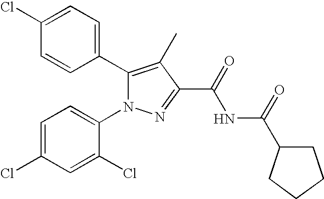

¹H NMR (400 MHz, CDCl₃) δ 9.33 (br s, 1H, —NH—), 7.44 (d, J=1.8 Hz, 1H), 7.34-7.24 (m, 4H), 7.06 (d, J=8.7 Hz, 2H), 3.74-3.66 (m, 1H), 2.38 (s, 3H), 2.06-1.97 (m, 2H), 1.95-1.86 (m, 2H), 1.80-1.71 (m, 2H), 1.69-1.60 (m, 2H). MH+ 476.

### Example 5

### 5-(4-Chlorophenyl)-N-(cyclohexanecarbonyl)-1-(2,4-dichlorophenyl)-4-methyl-1H-pyrazole-3-carboxamide

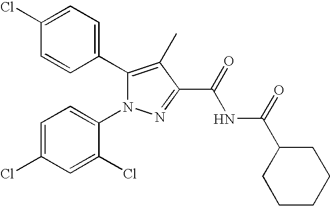

¹H NMR (400 MHz, CDCl₃) δ 9.31 (br s, 1H, —NH—), 7.44 (d, J=1.8 Hz, 1H), 7.34-7.24 (m, 4H), 7.06 (d, J=8.2 Hz, 2H), 3.29-3.21 (m, 1H), 2.38 (s, 3H), 2.03-1.96 (m, 2H), 1.86-1.78 (m, 2H), 1.75-1.68 (m, 1H), 1.56-1.33 (m, 4H), 1.31-1.20 (m, 1H). MH+ 490.

### Example 6

### N-Acetyl-5-(4-chlorophenyl)-1-(2,4-dichlorophenyl)-4-methyl-1H-pyrazole-3-carboxamide

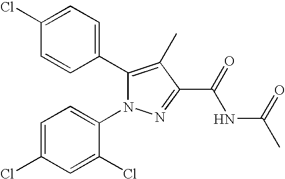

¹H NMR (400 MHz, CDCl₃) δ 9.39 (br s, 1H, —NH—), 7.45 (d, J=2.2 Hz, 1H), 7.34-7.24 (m, 4H), 7.06 (d, J=8.6 Hz, 2H), 2.60 (s, 3H), 2.38 (s, 3H). MH+ 422.

### Example 7

### N-Butyryl-5-(4-chlorophenyl)-1-(2,4-dichlorophenyl)-4-methyl-1H-pyrazole-3-carboxamide

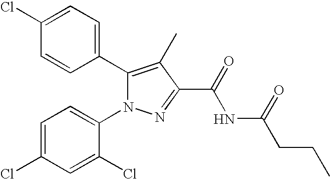

¹H NMR (400 MHz, CDCl₃) δ 9.36 (br s, 1H, —NH—), 7.45 (d, J=2.3 Hz, 1H), 7.34-7.24 (m, 4H), 7.06 (d, J=8.3 Hz, 2H), 2.94 (t, J=7.3 Hz, 2H), 2.37 (s, 3H), 1.81-1.71 (m, 2H), 1.03 (t, J=7.3 Hz, 3H). MH+ 450.

### Example 8

### 5-(4-Chlorophenyl)-1-(2,4-dichlorophenyl)-N-hexanoyl-4-methyl-1H-pyrazole-3-carboxamide

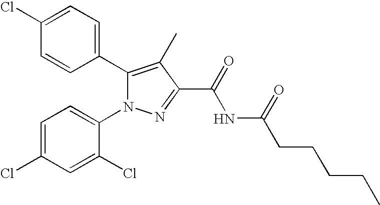

¹H NMR (400 MHz, CDCl₃) δ 9.35 (br s, 1H, —NH—), 7.44 (d, J=1.8 Hz, 1H), 7.34-7.24 (m, 4H), 7.06 (d, J=8.7 Hz, 2H), 2.96 (t, J=7.3 Hz, 2H), 2.37 (s, 3H), 1.77-1.69 (m, 2H), 1.42-1.33 (m, 4H), 0.91 (t, J=6.9 Hz, 3H). MH+ 478.

### Example 9

### 5-(4-Chlorophenyl)-1-(2,4-dichlorophenyl)-N-isobutyryl-4-methyl-1H-pyrazole-3-carboxamide

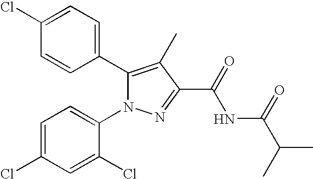

¹H NMR (400 MHz, CDCl₃) δ 9.34 (br s, 1H, —NH—), 7.45 (d, J=2.2 Hz, 1H), 7.34-7.24 (m, 4H), 7.06 (d, J=8.4 Hz, 2H), 3.58-3.48 (m, 1H), 2.38 (s, 3H), 1.26 (d, J=6.8 Hz, 6H). MH+ 450.

### Example 10

### 5-(4-Chlorophenyl)-1-(2,4-dichlorophenyl)-4-methyl-N-pivaloyl-1H-pyrazole-3-carboxamide

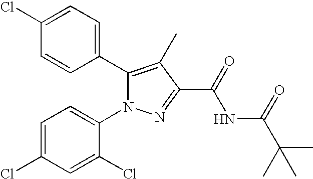

¹H NMR (400 MHz, CDCl₃) δ 9.80 (br s, 1H, —NH—), 7.46 (d, J=2.2 Hz, 1H), 7.34-7.22 (m, 4H), 7.06 (d, J=8.6 Hz, 2H), 2.38 (s, 3H), 1.29 (s, 9H). MH+ 464.

### Example 11

### 5-(4-Chlorophenyl)-1-(2,4-dichlorophenyl)-N-(2,2-dimethylbutanoyl)-4-methyl-1H-pyrazole-3-carboxamide

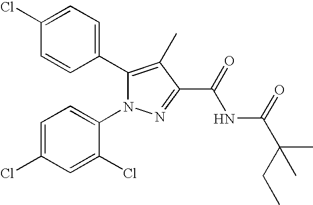

¹H NMR (400 MHz, CDCl₃) δ 9.79 (br s, 1H, —NH—), 7.47 (d, J=2.2 Hz, 1H), 7.34-7.22 (m, 4H), 7.07 (d, J=9.0 Hz, 2H), 2.38 (s, 3H), 1.64 (q, J=7.5 Hz, 2H), 1.25 (s, 6H), 0.92 (t, J=7.5 Hz, 3H). MH+ 478.

### Example 12

### 5-(4-Chlorophenyl)-1-(2,4-dichlorophenyl)-N-(3,3-dimethylbutanoyl)-4-methyl-1H-pyrazole-3-carboxamide

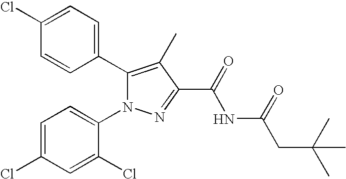

¹H NMR (400 MHz, CDCl₃) δ 9.32 (br s, 1H, —NH—), 7.45 (d, J=2.3 Hz, 1H), 7.34-7.24 (m, 4H), 7.06 (d, J=8.7 Hz, 2H), 2.86 (s, 2H), 2.37 (s, 3H), 1.12 (s, 9H). MH+ 478.

### Example 13

### 5-(4-Chlorophenyl)-1-(2,4-dichlorophenyl)-4-methyl-N-(2-propylpentanoyl)-1H-pyrazole-3-carboxamide

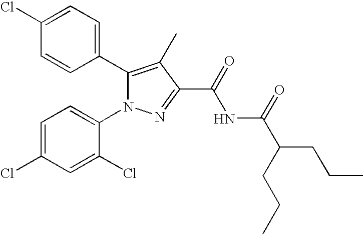

¹H NMR (400 MHz, CDCl₃) δ 9.37 (br s, 1H, —NH—), 7.45 (d, J=2.0 Hz, 1H), 7.35-7.24 (m, 4H), 7.06 (d, J=8.4 Hz, 2H), 3.45 (m, 1H), 2.38 (s, 3H), 1.81-1.68 (m, 2H), 1.60-1.30 (m, 6H), 0.92 (t, J=7.1 Hz, 6H). MH+ 506.

### Example 14

### 5-(4-Chlorophenyl)-1-(2,4-dichlorophenyl)-N-(2-ethylhexanoyl)-4-methyl-1H-pyrazole-3-carboxamide

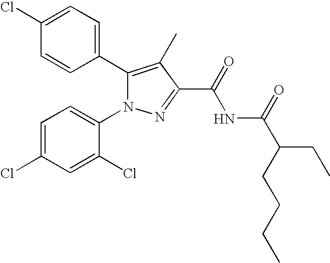

¹H NMR (400 MHz, CDCl₃) δ 9.38 (br s, 1H, —NH—), 7.45 (d, J=2.2 Hz, 1H), 7.36-7.25 (m, 4H), 7.06 (d, J=8.3 Hz, 2H), 3.35 (m, 1H), 2.38 (s, 3H), 1.82-1.69 (m, 2H), 1.66-1.47 (m, 2H), 1.42-1.26 (m, 4H), 0.97 (t, J=7.3 Hz, 3H), 0.89 (t, J=6.8 Hz, 3H). MH+ 506.

### Example 15

### 5-(4-Chlorophenyl)-1-(2,4-dichlorophenyl)-N-(hexylcarbamoyl)-4-methyl-1H-pyrazole-3-carboxamide

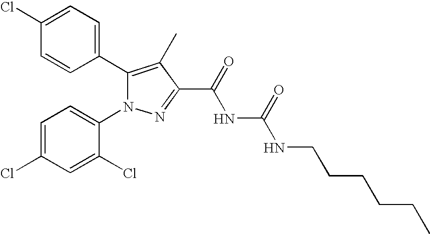

To a solution of 5-(4-chlorophenyl)-1-(2,4-dichlorophenyl)-4-methyl-1H-pyrazole-3-carboxamide (228 mg, 0.6 mmol) in THF (5 mL) was added 1M NaHMDS (0.9 mL, 0.9 mmol) at −78° C. under a nitrogen atmosphere. After stirring for 20 min, hexyl isocyanate (76.3 mg, 0.6 mmol) dissolved in THF (1 mL) was added dropwise thereto, and the mixture was reacted for 30 min. Then, the mixture was returned to room temperature and further reacted for 16 hr. After completion of the reaction, the reaction mixture was pour into saturated NaHCO₃ solution (30 mL) and extracted with EtOAc (50 mL). The organic layer was washed successively with water, dried over MgSO₄ and evaporated under vacuum. The residue was further purified by prep HPLC to provide the title compound (90.0 mg, 0.18 mmol, 30%) as a pale yellow solid. ¹H NMR (400 MHz, CDCl₃) 8.85 (br s, 1H, —NH—, imide), 8.36 (t, J=5.5 Hz, 1H, —NH—, amide), 7.44 (d, J=2.3 Hz, 1H), 7.34-7.23 (m, 4H), 7.06 (d, J=8.2 Hz, 2H), 3.35 (q, J=6.0 Hz, 2H), 2.36 (s, 3H), 1.64-1.56 (m, 2H), 1.42-1.29 (m, 6H), 0.90 (t, J=6.44 Hz, 3H). MH+ 507.

The following compounds of Examples 16 to 22 were obtained by repeating the procedure of Example 15.

### Example 16

### 5-(4-Chlorophenyl)-N-(cyclohexylcarbamoyl)-1-(2,4-dichlorophenyl)-4-methyl-1H-pyrazole-3-carboxamide

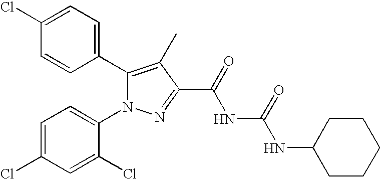

¹H NMR (400 MHz, CDCl₃) δ 8.83 (br s, 1H, —NH—, imide), 8.32 (d, J=8.2 Hz, 1H, —NH—, amide), 7.44 (d, J=2.3 Hz, 1H), 7.33-7.23 (m, 4H), 7.06 (d, J=8.2 Hz, 2H), 3.84-3.74 (m, 1H), 2.36 (s, 3H), 2.03-1.96 (m, 2H), 1.78-1.71 (m, 2H), 1.65-1.57 (m, 1H), 1.46-1.21 (m, 5H). MH+ 505.

### Example 17

### 5-(4-Chlorophenyl)-N-(cycloheptylcarbamoyl)-1-(2,4-dichlorophenyl)-4-methyl-1H-pyrazole-3-carboxamide

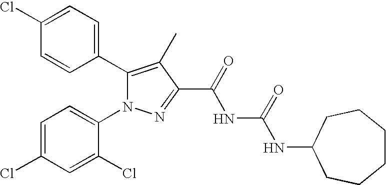

¹H NMR (400 MHz, CDCl₃) δ 8.81 (br s, 1H, —NH—, imide), 8.38 (d, J=7.8 Hz, 1H, —NH—, amide), 7.44 (d, J=2.3 Hz, 1H), 7.33-7.23 (m, 4H), 7.06 (d, J=8.7 Hz, 2H), 4.03-3.94 (m, 1H), 2.36 (s, 3H), 2.04-1.96 (m, 2H), 1.71-1.49 (m, 10H). MH+ 519.

### Example 18

### 5-(4-Chlorophenyl)-N-(cyclohexylmethylcarbamoyl)-1-(2,4-dichlorophenyl)-4-methyl-1H-pyrazole-3-carboxamide

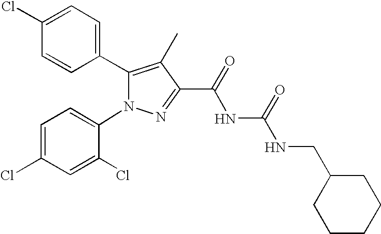

¹H NMR (400 MHz, CDCl₃) δ 8.86 (br s, 1H, —NH—, imide), 8.42 (t, J=5.9 Hz, 1H, —NH—, amide), 7.44 (d, J=1.8 Hz, 1H), 7.33-7.23 (m, 4H), 7.06 (d, J=8.2 Hz, 2H), 3.21 (t, J=6.4 Hz, 2H), 2.36 (s, 3H), 1.84-1.64 (m, 5H), 1.62-1.52 (m, 1H), 1.32-1.12 (m, 3H), 1.04-0.94 (m, 2H). MH+ 519.

### Example 19

### 5-(4-Chlorophenyl)-1-(2,4-dichlorophenyl)-N-(isopropylcarbamoyl)-4-methyl-1H-pyrazole-3-carboxamide

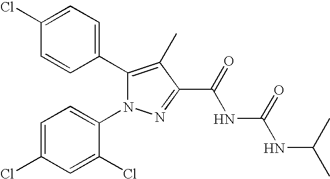

¹H NMR (400 MHz, CDCl₃) δ 8.82 (br s, 1H, —NH—, imide), 8.24 (d, J=7.8 Hz, 1H, —NH—, amide), 7.44 (d, J=2.3 Hz, 1H), 7.33-7.23 (m, 4H), 7.06 (d, J=8.7 Hz, 2H), 4.14-4.04 (m, 1H), 2.36 (s, 3H), 1.26 (d, J=6.4 Hz, 6H). MH+ 465.

### Example 20

### N-(tert-butylcarbamoyl)-5-(4-chlorophenyl)-1-(2,4-dichlorophenyl)-4-methyl-1H-pyrazole-3-carboxamide

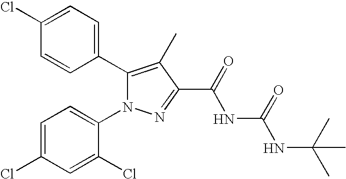

¹H NMR (400 MHz, CDCl₃) δ 8.70 (br s, 1H, —NH—, imide), 8.36 (br s, 1H, —NH—, amide), 7.44 (d, J=2.3 Hz, 1H), 7.33-7.23 (m, 4H), 7.06 (d, J=8.7 Hz, 2H), 2.35 (s, 3H), 1.44 (m, 9H). MH+ 479.

### Example 21

### 5-(4-Chlorophenyl)-1-(2,4-dichlorophenyl)-4-methyl-N-(phenylcarbamoyl)-1H-pyrazole-3-carboxamide

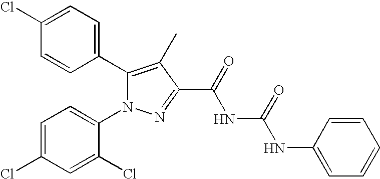

¹H NMR (400 MHz, CDCl₃) δ 10.53 (br s, 1H, —NH—, amide), 9.00 (br s, 1H, —NH—, imide), 7.59 (d, J=7.4 Hz, 2H), 7.46 (d, J=1.8 Hz, 1H), 7.37-7.28 (m, 6H), 7.14-7.06 (m, 3H), 2.40 (s, 3H). MH+ 499.

### Example 22

### N-(Benzylcarbamoyl)-5-(4-chlorophenyl)-1-(2,4-dichlorophenyl)-4-methyl-1H-pyrazole-3-carboxamide

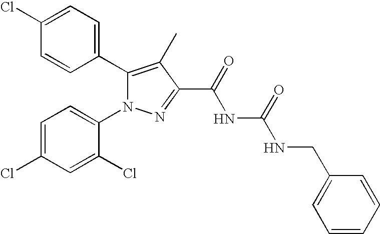

¹H NMR (400 MHz, CDCl₃) δ 8.94 (br s, 1H, —NH—, imide), 8.75 (t, J=5.5 Hz, 1H, —NH—, amide), 7.48-7.44 (m, 2H), 7.37-7.25 (m, 6H), 7.23-7.14 (m, 2H), 7.07-7.02 (m, 2H), 4.59-4.48 (m, 2H), 2.38 (s, 3H). MH+ 513.

### Example 23

Benzyl 5-(4-chlorophenyl)-1-(2,4-dichlorophenyl)-4-methyl-1H-pyrazole-3-carbonylcarbamate

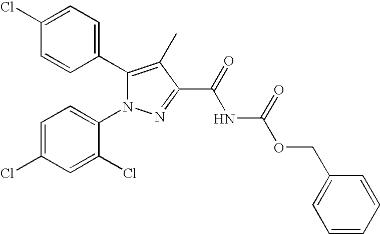

To a solution of -(4-chlorophenyl)-1-(2,4-dichlorophenyl)-4-methyl-1H-pyrazole-3-carboxamide (228 mg, 0.6 mmol) in THF (5 mL) was added 1M NaHMDS (0.9 mL, 0.9 mmol) at −78° C. under a nitrogen atmosphere. After stirring for 20 min, benzyl chloroformate (102 mg, 0.6 mmol) dissolved in THF (1 mL) was added dropwise thereto, and the mixture was reacted for 30 min. Then, the mixture was returned to room temperature and further reacted for 16 hr. After completion of the reaction, the reaction mixture was pour into saturated NaHCO₃ solution (30 mL) and extracted with EtOAc (50 mL). The organic layer was washed successively with water, dried over MgSO₄ and evaporated under vacuum. The residue was further purified by prep HPLC to provide the title compound (164 mg, 0.32 mmol, 53%) as a pale yellow solid.

¹H NMR (400 MHz, CDCl₃) δ 9.07 (br s, 1H, —NH—, imide), 7.45-7.24 (m, 10H), 7.06 (d, J=8.6 Hz, 2H), 5.26 (s, 2H), 2.37 (s, 3H). MH+ 514.

The following compounds of Examples 24 to 26 were obtained by repeating the procedure of Example 23.

### Example 24

### Butyl 5-(4-chlorophenyl)-1-(2,4-dichlorophenyl)-4-methyl-1H-pyrazole-3-carbonylcarbamate

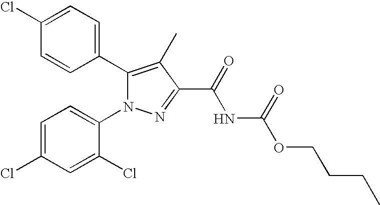

¹H NMR (400 MHz, CDCl₃) δ 8.99 (br s, 1H, —NH—, imide), 7.45 (d, J=2.2 Hz, 1H), 7.35-7.26 (m, 4H), 7.07 (d, J=8.4 Hz, 2H), 4.24 (t, J=6.8 Hz, 2H), 2.38 (s, 3H), 1.73-1.59 (m, 2H), 1.48-1.35 (m, 2H), 0.94 (t, J=7.3 Hz, 3H). MH+ 480.

### Example 25

### tert-Butyl 5-(4-chlorophenyl)-1-(2,4-dichlorophenyl)-4-methyl-1H-pyrazole-3-carbonylcarbamate

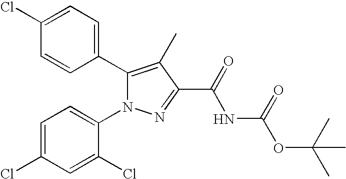

¹H NMR (400 MHz, CDCl₃) δ 8.85 (br s, 1H, —NH—, imide), 7.45 (d, J=2.0 Hz, 1H), 7.34-7.24 (m, 4H), 7.07 (d, J=8.8 Hz, 2H), 2.37 (s, 3H), 1.53 (s, 9H). MH+ 480.

### Example 26

### Neopentyl 5-(4-chlorophenyl)-1-(2,4-dichlorophenyl)-4-methyl-1H-pyrazole-3-carbonylcarbamate

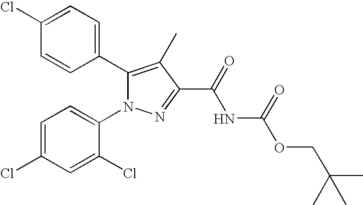

¹H NMR (400 MHz, CDCl₃) δ 8.98 (br s, 1H, —NH—, imide), 7.45 (d, J=1.8 Hz, 1H), 7.36-7.27 (m, 4H), 7.07 (d, J=8.4 Hz, 2H), 3.94 (s, 2H), 2.38 (s, 3H), 0.98 (s, 9H). MH+ 494.

### Preparation of 1,2-diaryl-N-acyl imidazole-4-carboxamide

### Preparation 2

### 1-(4-Chlorophenyl)-2-(2,4-dichlorophenyl)-5-methyl-1H-imidazole-4-carboxamide

To a suspension of 1-(4-chlorophenyl)-2-(2,4-dichlorophenyl)-5-methyl-1H-imidazole-4-carboxylic acid (286 mg, 0.75 mmol) in toluene (10 ml) was added thionyl chloride (0.273 ml, 3.75 mmol) and the mixture was refluxed for 3 hours and then cooled to the room temperature. The solvent was evaporated off under the reduced pressure. The residue was redissolved in toluene (10 ml) and the solvent was evaporated off again (procedure repeated twice) to yield the crude carboxyl chloride. Concentrated ammonium hydroxide solution (3 ml) was added dropwise to a solution of the carboxyl chloride obtained above in DCM (5 ml) at 0° C. The mixture was subsequently stirred at room temperature for 16 hours and then extracted with DCM (2×5 ml). The combined DCM was washed successively with water, dried over MgSO₄ and evaporated under vacuum to provide 281 mg (0.74 mmol, 98% yield) of the title compound as a yellow solid.

### Example 27

### 1-(4-Chlorophenyl)-2-(2,4-dichlorophenyl)-N-(2-ethylbutanoyl)-5-methyl-1H-imidazole-4-carboxamide

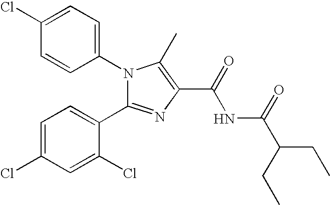

To a solution of 1-(4-chlorophenyl)-2-(2,4-dichlorophenyl)-5-methyl-1H-imidazole-4-carboxamide (95 mg, 0.25 mmol) in THF (2 mL) was added 1M NaHMDS (0.375 mL, 0.375 mmol) at −78° C. under a nitrogen atmosphere. After stirring for 20 min, 2-ethylbutyryl chloride (34 mg, 0.25 mmol) dissolved in THF (1 mL) was added dropwise thereto, and the mixture was reacted for 30 min. Then, the mixture was returned to room temperature and further reacted for 16 hr. After completion of the reaction, the reaction mixture was pour into saturated NaHCO₃ solution (10 mL) and extracted with EtOAc (20 mL). The organic layer was washed successively with water, dried over MgSO₄ and evaporated under vacuum. The residue was further purified by prep HPLC to provide the title compound (30 mg, 0.063 mmol, 25%) as a pale yellow solid.

¹H NMR (400 MHz, CDCl₃) δ 8.70 (br s, 1H, —NH—), 7.39-34 (m, 3H), 7.27 (s, 1H), 7.04 (d, J=9.2 Hz, 2H), 3.30-3.21 (m, 1H), 2.50 (s, 3H), 1.84-1.73 (m, 2H), 1.65-1.55 (m, 1H), 0.97 (t, J=7.3 Hz, 6H). MH+ 478.

The following compounds of Examples 28 to 32 were obtained by repeating the procedure of Example 27.

### Example 28

### 1-(4-Chlorophenyl)-N-(cyclohexanecarbonyl)-2-(2,4-dichlorophenyl)-5-methyl-1H-imidazole-4-carboxamide

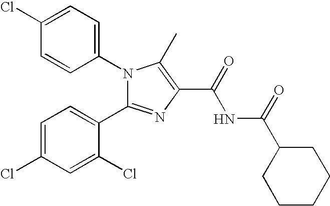

¹H NMR (400 MHz, CDCl₃) δ 9.63 (br s, 1H, —NH—), 7.39-7.34 (m, 3H), 7.27 (s, 1H), 7.05 (d, J=8.7 Hz, 2H), 3.26-3.17 (m, 1H), 2.49 (s, 3H), 2.03-1.96 (m, 2H), 1.86-1.78 (m, 2H), 1.74-1.68 (m, 1H), 1.56-1.46 (m, 2H), 1.44-1.20 (m, 3H). MH+ 490.

### Example 29

### 1-(4-Chlorophenyl)-2-(2,4-dichlorophenyl)-5-methyl-N-pivaloyl-1H-imidazole-4-carboxamide

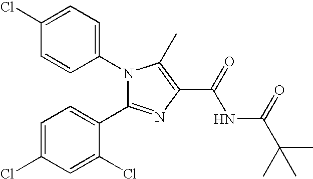

¹H NMR (400 MHz, CDCl₃) δ 10.18 (br s, 1H, —NH—), 7.37 (d, J=7.3 Hz, 3H), 7.25 (s, 1H), 7.04 (d, J=8.7 Hz, 2H), 2.49 (s, 3H), 1.31 (s, 9H). MH+ 464.

### Example 30

### 1-(4-Chlorophenyl)-2-(2,4-dichlorophenyl)-N-(2,2-dimethylbutanoyl)-5-methyl-1H-imidazole-4-carboxamide

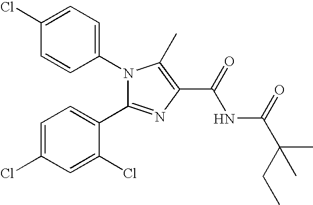

¹H NMR (400 MHz, CDCl₃) δ 10.17 (br s, 1H, —NH—), 7.38 (d, J=6.6 Hz, 3H), 7.27 (s, 1H), 7.05 (d, J=8.8 Hz, 2H), 2.49 (s, 3H), 1.66 (q, J=7.3 Hz, 2H), 1.26 (s, 6H), 0.93 (t, J=7.3 Hz, 3H). MH+ 478.

### Example 31

### 1-(4-Chlorophenyl)-2-(2,4-dichlorophenyl)-5-methyl-N-(2-propylpentanoyl)-1H-imidazole-4-carboxamide

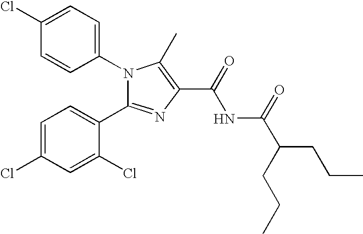

¹H NMR (400 MHz, CDCl₃) δ 9.69 (br s, 1H, —NH—), 7.37 (d, J=8.8 Hz, 3H), 7.27 (s, 1H), 7.04 (d, J=8.6 Hz, 2H), 3.49-3.35 (m, 1H), 2.50 (s, 3H), 1.81-1.70 (m, 2H), 1.55-1.32 (m, 6H), 0.91 (t, J=7.1 Hz, 6H). MH+ 506.

### Example 32

### 1-(4-Chlorophenyl)-2-(2,4-dichlorophenyl)-N-(2-ethylhexanoyl)-5-methyl-1H-imidazole-4-carboxamide

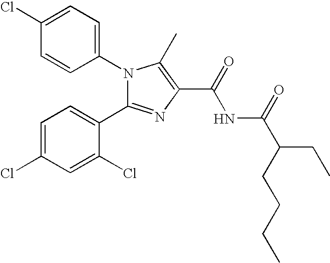

¹H NMR (400 MHz, CDCl₃) δ 9.70 (br s, 1H, —NH—), 7.37 (d, J=9.9 Hz, 3H), 7.27 (s, 1H), 7.05 (d, J=8.8 Hz, 2H), 3.38-3.27 (m, 1H), 2.50 (s, 3H), 1.83-1.70 (m, 2H), 1.65-1.47 (m, 2H), 1.38-1.26 (m, 4H), 0.97 (t, J=7.5 Hz, 3H), 0.88 (t, J=7.0 Hz, 3H). MH+ 506.

### Preparation of N-aminomethyl-1,5-diaryl pyrazole-3-carboxamide

### Preparation 3

### N-(Aminomethyl)-5-(4-chlorophenyl)-1-(2,4-dichlorophenyl)-4-methyl-1H-pyrazole-3-carboxamide

To a mixture of glycinamide hydrochloric acid (166 mg, 1.5 mmol), HOBt (243 mg, 1.8 mmol), NMM (1.82 g, 18 mmol) and 5-(4-chlorophenyl)-1-(2,4-dichlorophenyl)-4-methyl-1H-pyrazole-3-carboxylic acid (572 mg, 1.5 mmol) in DMF (15 mL) was added EDCI (345 mg, 1.8 mmol). The reaction mixture was subsequently stirred at room temperature overnight and then the solvent was evaporated off under the reduced pressure. The residue was redissolved in DCM (30 ml) and then washed successively with water, dried over MgSO₄ and evaporated under vacuum to provide 550 mg (1.26 mmol, 84% yield) of N-carbamoylmethyl amide (19) as an intermediate. PIFA (320 mg, 0.74 mmol) was dissolved in ACN (1.9 mL). To this solution water (1.9 mL) was added. Finally 310 mg (0.71 mmol) of N-carbamoylmethyl amide (19) was added, and the mixture was stirred at room temperature overnight. The mixture was diluted with 1M HCl (20 mL) and then washed with ether (2×20 mL). The aqueous layer was concentrated under vacuum to provide 108 mg (0.24 mmol, 33% yield) of title compound. The resulting residue was used in the next step without further purification.

### Example 33

### 5-(4-Chlorophenyl)-1-(2,4-dichlorophenyl)-4-methyl-N-(octanamidomethyl)-1H-pyrazole-3-carboxamide

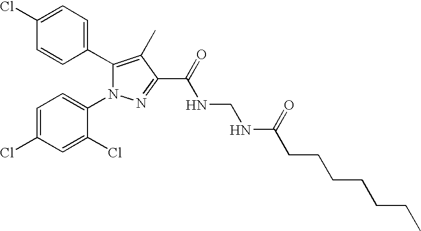

To a mixture of octanoic acid (32.8 mg, 0.23 mmol), DMAP (55.5 mg, 0.46 mmol) and N-(aminomethyl)-5-(4-chlorophenyl)-1-(2,4-dichlorophenyl)-4-methyl-1H-pyrazole-3-carboxamide hydrochloric acid salt (92.2 mg, 0.21 mmol) in DCM (5 mL) was added EDCI (43.6 mg, 0.23 mmol). After stirring at room temperature overnight, the reaction mixture was pour into 1M HCl solution (10 mL) and extracted with DCM (2×20 mL). The combined organic layer was washed successively with water, dried over MgSO₄ and evaporated under vacuum. The residue was further purified by prep HPLC to provide the title compound (60 mg, 0.11 mmol, 53%) as pale yellow solid.

¹H NMR (400 MHz, CDCl₃) δ 7.80 (br t, J=6.4 Hz, 1H, —NH—, amide), 7.41 (d, J=2.3 Hz, 1H), 7.31-7.27 (m, 4H), 7.05 (d, J=8.7 Hz, 2H), 6.56 (br t, J=6.0 Hz, 1H, —NH—, amide), 4.80 (t, J=6.4 Hz, 2H), 2.35 (s, 3H), 2.17 (t, J=7.8 Hz, 2H), 1.64-1.57 (m, 2H), 1.31-1.20 (m, 8H), 0.85 (t, J=6.4 Hz, 3H). MH+ 535.

The following compounds of Examples 34 to 41 were obtained by repeating the procedure of Example 33.

### Example 34

### 5-(4-Chlorophenyl)-N-(cyclobutanecarboxamidomethyl)-1-(2,4-dichlorophenyl)-4-methyl-1H-pyrazole-3-carboxamide

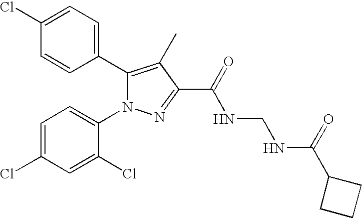

¹H NMR (400 MHz, CDCl₃) δ 7.80 (br t, J=5.9 Hz, 1H, —NH—, amide), 7.42 (d, J=2.3 Hz, 1H), 7.32-7.25 (m, 4H), 7.05 (d, J=8.7 Hz, 2H), 6.45 (br t, J=6.4 Hz, 1H, —NH—, amide), 4.81 (t, J=6.4 Hz, 2H), 3.03-2.95 (m, 1H), 2.36 (s, 3H), 2.32-2.21 (m, 2H), 2.18-2.09 (m, 2H), 2.01-1.81 (m, 2H). MH+ 491.

### Example 35

### 5-(4-Chlorophenyl)-N-(cyclohexanecarboxamidomethyl)-1-(2,4-dichlorophenyl)-4-methyl-1H-pyrazole-3-carboxamide

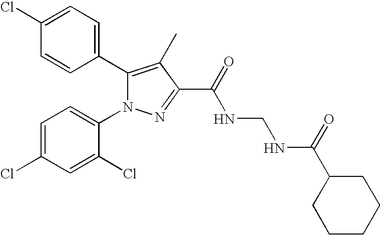

¹H NMR (400 MHz, CDCl₃) δ 7.79 (br t, J=6.4 Hz, 1H, —NH—, amide), 7.42 (d, J=2.3 Hz, 1H), 7.32-7.25 (m, 4H), 7.05 (d, J=8.3 Hz, 2H), 6.58 (br t, J=6.4 Hz, 1H, —NH—, amide), 4.81 (t, J=6.4 Hz, 2H), 2.36 (s, 3H), 2.11-2.03 (m, 1H), 1.88-1.62 (m, 6H), 1.47-1.36 (m, 2H), 1.30-1.16 (m, 2H). MH+ 519.

### Example 36

### N-(Butyramidomethyl)-5-(4-chlorophenyl)-1-(2,4-dichlorophenyl)-4-methyl-1H-pyrazole-3-carboxamide

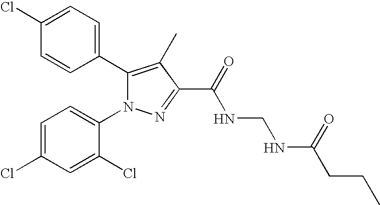

¹H NMR (400 MHz, CDCl₃) δ 7.80 (br t, J=6.4 Hz, 1H, —NH—, amide), 7.42 (d, J=1.8 Hz, 1H), 7.32-7.24 (m, 4H), 7.05 (d, J=8.7 Hz, 2H), 6.55 (br t, J=6.0 Hz, 1H, —NH—, amide), 4.81 (t, J=6.4 Hz, 2H), 2.36 (s, 3H), 2.16 (t, J=7.3 Hz, 2H), 1.70-1.62 (m, 2H), 0.93 (t, J=7.3 Hz, 3H). MH+ 479.

### Example 37

### 5-(4-Chlorophenyl)-1-(2,4-dichlorophenyl)-N-(isobutyramidomethyl)-4-methyl-1H-pyrazole-3-carboxamide

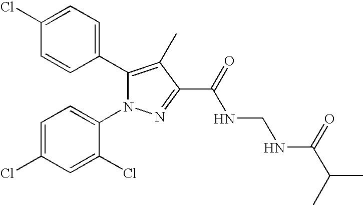

¹H NMR (400 MHz, CDCl₃) δ 7.80 (br t, J=6.0 Hz, 1H, —NH—, amide), 7.42 (d, J=1.8 Hz, 1H), 7.32-7.24 (m, 4H), 7.05 (d, J=8.7 Hz, 2H), 6.58 (br t, J=5.9 Hz, 1H, —NH—, amide), 4.82 (t, J=6.4 Hz, 2H), 2.39-2.32 (m, 1H), 2.36 (s, 3H), 1.15 (d, J=6.9 Hz, 6H). MH+ 479.

### Example 38

### 5-(4-Chlorophenyl)-N-(cycloheptanecarboxamidomethyl)-1-(2,4-dichlorophenyl)-4-methyl-1H-pyrazole-3-carboxamide

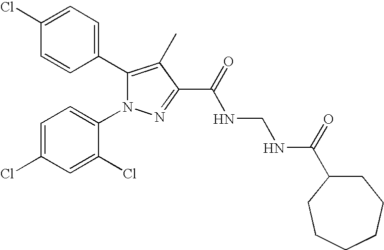

¹H NMR (400 MHz, CDCl₃) δ 7.79 (br t, J=6.4 Hz, 1H, —NH—, amide), 7.42 (d, J=1.8 Hz, 1H), 7.32-7.25 (m, 4H), 7.05 (d, J=8.7 Hz, 2H), 6.52 (br t, J=6.4 Hz, 1H, —NH—, amide), 4.80 (t, J=6.9 Hz, 2H), 2.36 (s, 3H), 2.25-2.16 (m, 1H), 1.90-1.83 (m, 2H), 1.78-1.69 (m, 2H), 1.68-1.38 (m, 8H). MH+ 533.

### Example 39

### 5-(4-Chlorophenyl)-N-((2-cyclohexylacetamido)methyl)-1-(2,4-dichlorophenyl)-4-methyl-1H-pyrazole-3-carboxamide

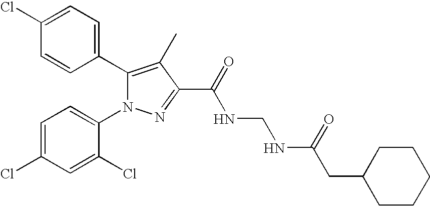

¹H NMR (400 MHz, CDCl₃) δ 7.81 (br t, J=6.4 Hz, 1H, —NH—, amide), 7.42 (d, J=2.3 Hz, 1H), 7.32-7.24 (m, 4H), 7.05 (d, J=8.7 Hz, 2H), 6.55 (br t, J=5.9 Hz, 1H, —NH—, amide), 4.80 (t, J=6.4 Hz, 2H), 2.36 (s, 3H), 2.04 (d, J=6.8 Hz, 2H), 1.82-1.61 (m, 5H), 1.31-1.19 (m, 2H), 1.17-1.06 (m, 2H), 0.98-0.87 (m, 2H). MH+ 533.

### Example 40

### 5-(4-Chlorophenyl)-1-(2,4-dichlorophenyl)-4-methyl-N-((3-methylcyclohexanecarboxamido)methyl)-1H-pyrazole-3-carboxamide

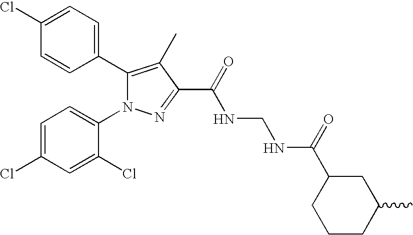

¹H NMR (400 MHz, CDCl₃) δ 7.79 (br t, J=6.4 Hz, 1H, —NH—, amide), 7.42 (d, J=1.8 Hz, 1H), 7.32-7.24 (m, 4H), 7.05 (d, J=8.7 Hz, 2H), 6.56 (br t, J=6.0 Hz, 1H, —NH—, amide), 4.84-4.78 (m, 2H), 2.36 (s, 3H), 2.13-2.06 (m, 1H), 1.88-1.75 (m, 4H), 1.66-1.63 (m, 1H), 1.43-1.16 (m, 2H), 1.11-1.02 (m, 1H), 0.90 (d, J=6.4 Hz, 3H), 0.89-0.81 (m, 1H). MH+ 533.

### Example 41

### 5-(4-Chlorophenyl)-N-((2-cyclopentylacetamido)methyl)-1-(2,4-dichlorophenyl)-4-methyl-1H-pyrazole-3-carboxamide

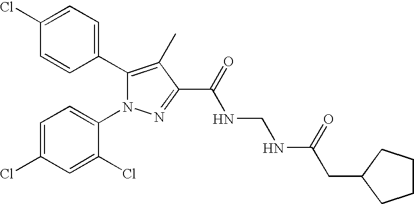

¹H NMR (400 MHz, CDCl₃) δ 7.82 (br t, J=6.4 Hz, 1H, —NH—, amide), 7.42 (d, J=2.3 Hz, 1H), 7.32-7.24 (m, 4H), 7.05 (d, J=8.7 Hz, 2H), 6.62 (br t, J=6.4 Hz, 1H, —NH—, amide), 4.81 (t, J=6.4 Hz, 2H), 2.36 (s, 3H), 2.23-2.17 (m, 3H), 1.86-1.77 (m, 2H), 1.65-1.48 (m, 4H), 1.19-1.08 (m, 2H). MH+ 519.

Pharmacological Test In vitro Activity Analysis

The compounds of the present invention were analyzed for their binding characteristics for CB₁ and CB₂ and the pharmacological activity thereof in accordance with the method disclosed in [Devane W A, Dysarz F A 3^{rd}, Johnson M R, Melvin L S and Howlett A C, Determination and characterization of a cannabinoid receptor in rat brain, Mol. Pharmacol., 34(5): 605-13 (1998)]. The analysis was performed using [³H]CP-55940 which is a selectively radioactivity-labeled 5-(1,1-dimethyheptyl)-2[5-hydroxy-2-(3-hydroxypropyl)-cyclohexyl]-phenol, purchased from PerkinElmer Life Sciences, Inc. (Boston, Mass., U.S.A.), through a rat CB-1 receptor binding protocol as follows.

The tissue obtained from the brain of SD rats was homogenized with a Dounce homogenate system in TME (50 mM Tris, 3 mM MgCl₂ and 1 mM EDTA, pH 7.4) at 4° C., and the homogenate was centrifuged at 48,000 g for 30 min. at 4° C. The pellet was resuspended in 5 ml of TME and the suspension was divided into aliquots and stored at −70° C. until its use in the following assay.

2 μl of the test compound was diluted in dimethylsulphoxide and was added to a deep well of a polypropylene plate, to which 50 μl of [³H]CP-55940 diluted in a ligand buffer solution (0.1% bovine serum albumin(BAS)+TME) was added. The tissue concentrations were determined by Bradford protein analysis, and 148 μl of brain tissue of the required concentration was added to the plate. The plate was covered and placed in a 30° C. incubator for 60 min, and then transformed on GF/B filtermat pretreated in polyethylenimine (PEI) using a cell harvester. Each filter was washed five times and dried at 60° C. for 1 hr. Then, the degree of radioactivity retained by the filter was measured using Wallac Microbeta™ (PerkinElmer Life Sciences, Inc., Massachusetts, U.S.A.) and the activity of the compound for inhibiting CB₁ receptor was determined therefrom.

While the invention has been described with respect to the specific embodiments, it should be recognized that various modifications and changes may be made by those skilled in the art to the invention which also fall within the scope of the invention as defined as the appended claims.

What is claimed is:

1. A compound of formula (I) or a pharmaceutically acceptable salt thereof: 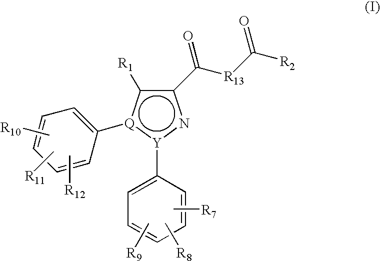
wherein:
Q is carbon and Y is nitrogen, or Q is nitrogen and Y is carbon;
R₁ is hydrogen, halogen, C₁₋₇ alkyl, substituted C₁₋₇ alkyl, C₂₋₇ alkenyl, substituted C₂₋₇ alkenyl, C₂₋₇ alkynyl, substituted C₂₋₇ alkynyl, or (CH₂)ₙ—C₃₋₅ carbocycle, n being 0 or 1;
R₂ is hydrogen, OR₃, NR₄R₅, C₁₋₇ alkyl, substituted C₁₋₇ alkyl, C₂₋₇ alkenyl, substituted C₂₋₇ alkenyl, C₂₋₇ alkynyl, substituted C₂₋₇ alkynyl, C₃₋₇ cycloalkyl, substituted C₃₋₇ cycloalkyl, aryl, substituted aryl, heteroaryl, substituted heteroaryl, heterocycloalkyl, substituted heterocycloalkyl; C₁₋₈ alkyl optionally substituted by alkoxy or halogen, C₂₋₆ alkenyl optionally substituted by alkoxy or halogen, (CH₂)ₘ—C₃₋₆ carbocycle optionally substituted by alkoxy or halogen, or (CH₂)ₘ—R₆, m being 1 or 2;
R₃ is C₁₋₇ alkyl, substituted C₁₋₇ alkyl, C₂₋₇ alkenyl, substituted C₂₋₇ alkenyl, C₃₋₇ cycloalkyl, substituted C₃₋₇ cycloalkyl, aryl, substituted aryl, heteroaryl, substituted heteroaryl, heterocycloalkyl, substituted heterocycloalkyl; or
R₄ and R₅ are each independently hydrogen, C₁₋₆ alkyl, substituted C₁₋₆ alkyl, C₂₋₆ alkenyl, substituted C₂₋₆ alkenyl, C₃₋₇ cycloalkyl, substituted C₃₋₇ cycloalkyl, aryl, substituted aryl, heteroaryl, substituted heteroaryl, heterocycloalkyl, substituted heterocycloalkyl; or
R₄ and R₅, together with the nitrogen atom to which they are bonded, form a 4- to 10-membered saturated or unsaturated heterocyclic ring which is optionally substituted with one or more C₁₋₃ alkyl, benzyl, phenyl, C₁₋₃ alkoxy or halogen;
R₆ is phenyl, furanyl, benzofuranyl, thienyl, benzothienyl, pyridinyl, pyrimidinyl, pyrazinyl, pyridizinyl, tetrahydrofuranyl, tetrahydropyranyl, dioxanyl, 1,4-benzodioxanyl or benzo[1,3]dioxolyl, each being optionally substituted by one or more groups consisting of halogen, C₁₋₃ alkyl and C₁₋₂ alkoxy, each having optional one to three fluorine substitutes;
R₇, R₈, R₉, R₁₀, R₁₁ and R₁₂ are each independently hydrogen, halogen, cyano, C₁₋₃ alkyl, C₁₋₃ alkoxy or trifluoromethyl; and

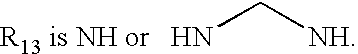

2. The compound of claim 1, which is a compound of formula (Ia), (Ib) or (Ic): 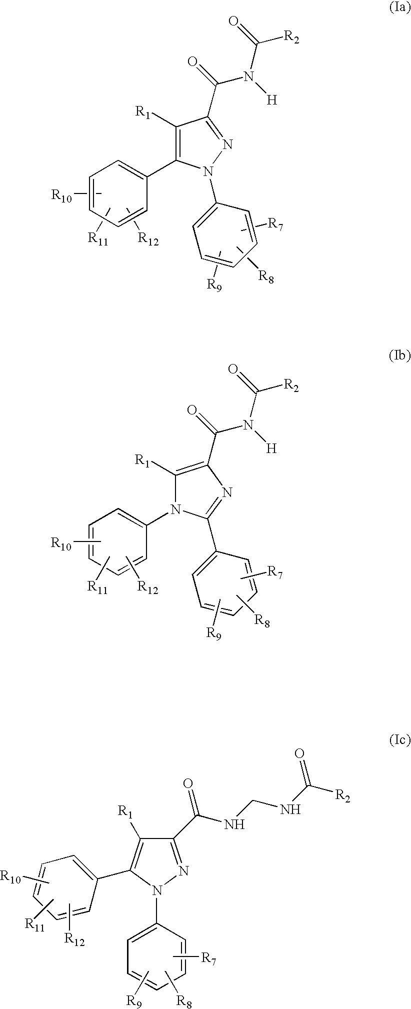
wherein, R₁, R₂, R₇, R₈, R₉, R₁₀, R₁₁, and R₁₂ have the same meanings as defined in claim 1.

3. The compound of claim 2, wherein in the compound of formula (Ic), R₂ is hydrogen, optionally substituted C₁₋₇ alkyl, optionally substituted C₂₋₇ alkenyl, optionally substituted C₂₋₇ alkynyl, optionally substituted C₃₋₇ cycloalkyl, optionally substituted aryl, optionally substituted heteroaryl, or optionally substituted heterocycloalkyl.

4. The compound of claim 1, which is selected from the group consisting of:
5-(4-Chlorophenyl)-1-(2,4-dichlorophenyl)-N-(2-ethylbutanoyl)-4-methyl-1H-pyrazole-3-carboxamide;
5-(4-Chlorophenyl)-N-(cyclopropanecarbonyl)-1-(2,4-dichlorophenyl)-4-methyl-1H-pyrazole-3-carboxamide;
5-(4-Chlorophenyl)-N-(cyclobutanecarbonyl)-1-(2,4-dichlorophenyl)-4-methyl-1H-pyrazole-3-carboxamide;
5-(4-Chlorophenyl)-N-(cyclopentanecarbonyl)-1-(2,4-dichlorophenyl)-4-methyl-1H-pyrazole-3-carboxamide;
5-(4-Chlorophenyl)-N-(cyclohexanecarbonyl)-1-(2,4-dichlorophenyl)-4-methyl-1H-pyrazole-3-carboxamide;
N-Acetyl-5-(4-chlorophenyl)-1-(2,4-dichlorophenyl)-4-methyl-1H-pyrazole-3-carboxamide;
N-Butyryl-5-(4-chlorophenyl)-1-(2,4-dichlorophenyl)-4-methyl-1H-pyrazole-3-carboxamide;
5-(4-Chlorophenyl)-1-(2,4-dichlorophenyl)-N-hexanoyl-4-methyl-1H-pyrazole-3-carboxamide;
5-(4-Chlorophenyl)-1-(2,4-dichlorophenyl)-N-isobutyryl-4-methyl-1H-pyrazole-3-carboxamide;
5-(4-Chlorophenyl)-1-(2,4-dichlorophenyl)-4-methyl-N-pivaloyl-1H-pyrazole-3-carboxamide;
5-(4-Chlorophenyl)-1-(2,4-dichlorophenyl)-N-(2,2-dimethylbutanoyl)-4-methyl-1H-pyrazole-3-carboxamide;
5-(4-Chlorophenyl)-1-(2,4-dichlorophenyl)-N-(3,3-dimethylbutanoyl)-4-methyl-1H-pyrazole-3-carboxamide;
5-(4-Chlorophenyl)-1-(2,4-dichlorophenyl)-4-methyl-N-(2-propylpentanoyl)-1H-pyrazole-3-carboxamide;
5-(4-Chlorophenyl)-1-(2,4-dichlorophenyl)-N-(2-ethylhexanoyl)-4-methyl-1H-pyrazole-3-carboxamide;
5-(4-Chlorophenyl)-1-(2,4-dichlorophenyl)-N-(hexylcarbamoyl)-4-methyl-1H-pyrazole-3-carboxamide;
5-(4-Chlorophenyl)-N-(cyclohexylcarbamoyl)-1-(2,4-dichlorophenyl)-4-methyl-1H-pyrazole-3-carboxamide;
5-(4-Chlorophenyl)-N-(cycloheptylcarbamoyl)-1-(2,4-dichlorophenyl)-4-methyl-1H-pyrazole-3-carboxamide;
5-(4-Chlorophenyl)-N-(cyclohexylmethylcarbamoyl)-1-(2,4-dichlorophenyl)-4-methyl-1H-pyrazole-3-carboxamide;
5-(4-Chlorophenyl)-1-(2,4-dichlorophenyl)-N-(isopropylcarbamoyl)-4-methyl-1H-pyrazole-3-carboxamide;
N-(tert-butylcarbamoyl)-5-(4-chlorophenyl)-1-(2,4-dichlorophenyl)-4-methyl-1H-pyrazole-3-carboxamide;
5-(4-Chlorophenyl)-1-(2,4-dichlorophenyl)-4-methyl-N-(phenylcarbamoyl)-1H-pyrazole-3-carboxamide;
N-(Benzylcarbamoyl)-5-(4-chlorophenyl)-1-(2,4-dichlorophenyl)-4-methyl-1H-pyrazole-3-carboxamide;
Benzyl 5-(4-chlorophenyl)-1-(2,4-dichlorophenyl)-4-methyl-1H-pyrazole-3-carbonylcarbamate;
Butyl 5-(4-chlorophenyl)-1-(2,4-dichlorophenyl)-4-methyl-1H-pyrazole-3-carbonylcarbamate;
tert-Butyl 5-(4-chlorophenyl)-1-(2,4-dichlorophenyl)-4-methyl-1H-pyrazole-3-carbonylcarbamate;
Neopentyl 5-(4-chlorophenyl)-1-(2,4-dichlorophenyl)-4-methyl-1H-pyrazole-3-carbonylcarbamate;
1-(4-Chlorophenyl)-2-(2,4-dichlorophenyl)-N-(2-ethylbutanoyl)-5-methyl-1H-imidazole-4-carboxamide;
1-(4-Chlorophenyl)-N-(cyclohexanecarbonyl)-2-(2,4-dichlorophenyl)-5-methyl-1H-imidazole-4-carboxamide;
1-(4-Chlorophenyl)-2-(2,4-dichlorophenyl)-5-methyl-N-pivaloyl-1H-imidazole-4-carboxamide;
1-(4-Chlorophenyl)-2-(2,4-dichlorophenyl)-N-(2,2-dimethylbutanoyl)-5-methyl-1H-imidazole-4-carboxamide;
1-(4-Chlorophenyl)-2-(2,4-dichlorophenyl)-5-methyl-N-(2-propylpentanoyl)-1H-imidazole-4-carboxamide;
1-(4-Chlorophenyl)-2-(2,4-dichlorophenyl)-N-(2-ethylhexanoyl)-5-methyl-1H-imidazole-4-carboxamide;
5-(4-Chlorophenyl)-1-(2,4-dichlorophenyl)-4-methyl-N-(octanamidomethyl)-1H-pyrazole-3-carboxamide;
5-(4-Chlorophenyl)-N-(cyclobutanecarboxamidomethyl)-1-(2,4-dichlorophenyl)-4-methyl-1H-pyrazole-3-carboxamide;
5-(4-Chlorophenyl)-N-(cyclohexanecarboxamidomethyl)-1-(2,4-dichlorophenyl)-4-methyl-1H-pyrazole-3-carboxamide;
N-(Butyramidomethyl)-5-(4-chlorophenyl)-1-(2,4-dichlorophenyl)-4-methyl-1H-pyrazole-3-carboxamide;
5-(4-Chlorophenyl)-1-(2,4-dichlorophenyl)-N-(isobutyramidomethyl)-4-methyl-1H-pyrazole-3-carboxamide;
5-(4-Chlorophenyl)-N-(cycloheptanecarboxamidomethyl)-1-(2,4-dichlorophenyl)-4-methyl-1H-pyrazole-3-carboxamide;
5-(4-Chlorophenyl)-N-((2-cyclohexylacetamido)methyl)-1-(2,4-dichlorophenyl)-4-methyl-1H-pyrazole-3-carboxamide;
5-(4-Chlorophenyl)-1-(2,4-dichlorophenyl)-4-methyl-N-((3-methylcyclohexanecarboxamido)methyl)-1H-pyrazole-3-carboxamide; and
5-(4-Chlorophenyl)-N-((2-cyclopentylacetamido)methyl)-1-(2,4-dichlorophenyl)-4-methyl-1H-pyrazole-3-carboxamide.

5. A method for preparing the compound of formula (Ia-1), which comprises (i) converting a carboxylic acid derivative of formula (5) to a carbonyl chloride compound of formula (6); (ii) subjecting the carbonyl chloride compound of formula (6) to amination with an ammonium hydroxide to obtain an amide compound of formula (7); and (iii) coupling the amide compound of formula (7) with an acyl chloride compound of formula (8) in the presence of a base: 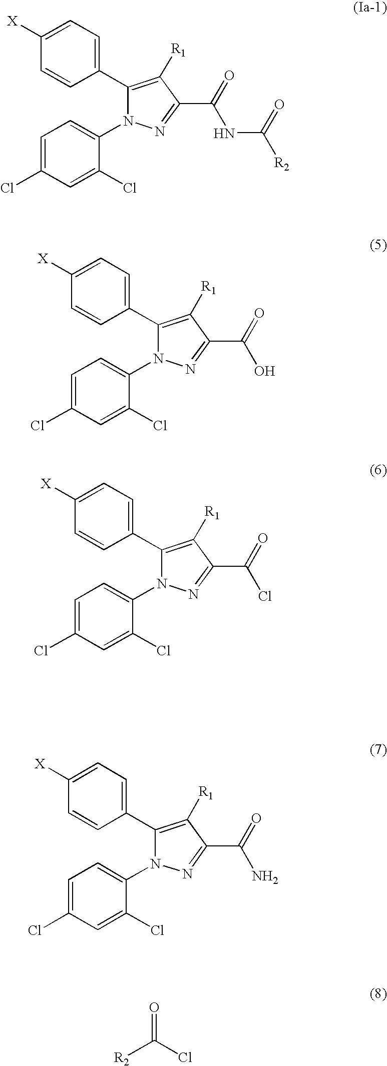
wherein R₁, and R₂ have the same meanings as defined in claim 1 above; and X is halogen.

6. A method for preparing the compound of formula (Ib-1), which comprises (i) converting a carboxylic acid derivative of formula (15) to a carbonyl chloride compound of formula (16); (ii) subjecting the carbonyl chloride compound of formula (16) to amination with an ammonium hydroxide to obtain an amide compound of formula (17); and (iii) coupling the amide compound of formula (17) with an acyl chloride compound of formula (8) in the presence of a base: 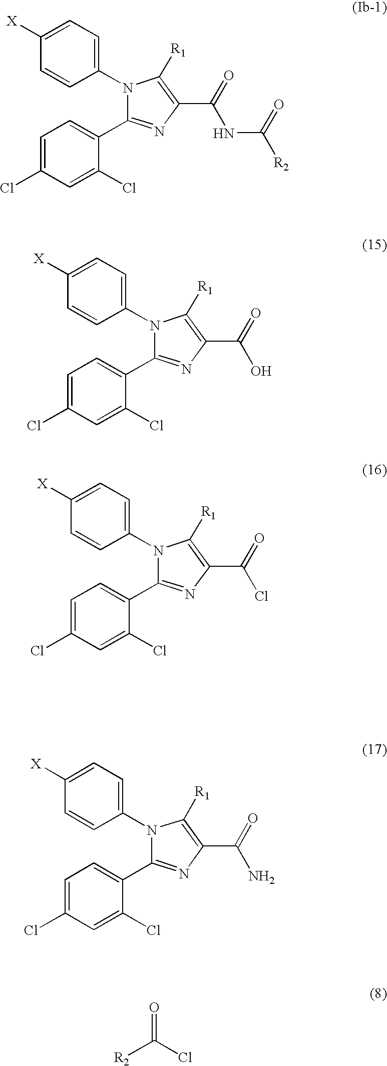
wherein R₁, and R₂ have the same meanings as defined in claim 1 above; and X is halogen.

7. A method for preparing the compound of formula (Ic-1), which comprises (i) coupling a carboxylic acid derivative of formula (5) with a glycinamide hydrochloride in the presence of a coupling agent to obtain a N-carbamoylmethyl amide compound of formula (19); (ii) reacting the N-carbamoylmethyl amide compound of formula (19) with [bis(trifluoroacetoxy)iodo]-benzene (PIFA) or iodine in a solvent to obtain a N-aminomethyl amide hydrochloric acid salt of formula (20); and (iii) coupling the N-aminomethyl amide hydrochloric acid salt of formula (20) with a compound of formula (21) in the presence of a coupling agent: 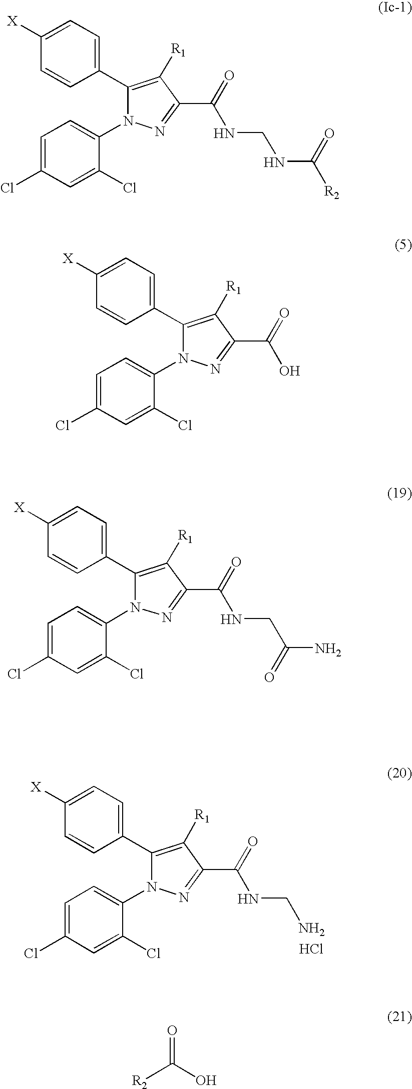
wherein R₁, and R₂ have the same meanings as defined in claim 1 above; and X is halogen.

8. A pharmaceutical composition comprising the compound of formula (I) of claim 1 as an active ingredient and a pharmaceutically acceptable carrier.

9. A method for preventing or treating obesity or an obesity-related metabolic disorder in a mammal, which comprises administering the compound of formula (I) of claim 1 to the mammal.

10. A method for inhibiting cannabinoid CB₁ receptor in a mammal, which comprises administering the compound of formula (I) of claim 1 to the mammal.
